(19) United States
(12) Patent Application Publication
(10) Pub. No.: US 2022/0331097 A1
(43) Pub. Date: Oct. 20, 2022

(54) Apparatus and Methods for Valve Tether Tensioning and Fixation

(71) Applicant: Tendyne Holdings, Inc., St. Paul, MN (US)

(72) Inventors: Alec King, Maple Grove, MN (US); Amy Marie Danielson, White Bear Lake, MN (US); David A. Panus, Maple Grove, MN (US); Chase Carlson, Lakeland, MN (US)

(73) Assignee: Tendyne Holdings, Inc., St. Paul, MN (US)

(21) Appl. No.: 17/703,310

(22) Filed: Mar. 24, 2022

Related U.S. Application Data
(60) Provisional application No. 63/176,400, filed on Apr. 19, 2021.

(51) Int. Cl. A61F 2/24

(52) U.S. Cl. CPC A61F 2/2418; A61F 2/2436; A61F 2220/0016

(57) ABSTRACT

A prosthetic heart valve system includes a prosthetic heart valve, an anchor, a tether, and a tensioning member. The prosthetic heart valve has an expandable stent and a prosthetic valve assembly disposed within the stent. The anchor is adapted to be disposed on or adjacent an epicardial surface of a heart of a patient. The tether has a first end coupled to the anchor and a second end coupled to the prosthetic heart valve. The tensioning member is disposed along an intermediate portion of the tether between the first end of the tether and the second end of the tether, the tether forming a loop around the tensioning member. The tensioning member is adjustable to increase or decrease a size of the loop to change an effective length of the tether between the prosthetic heart valve and the anchor.

### CROSS-REFERENCE TO RELATED APPLICATIONS

The present application claims the benefit of the filing date of U.S. Provisional Patent Application No. 63/176,400 filed Apr. 19, 2021, the disclosure of which is hereby incorporated herein by reference.

### BACKGROUND OF THE DISCLOSURE

Valvular heart disease, and specifically aortic and mitral valve disease, is a significant health issue in the United States. Valve replacement is one option for treating heart valves diseases. Traditional valve replacement surgery, the orthotopic replacement of a heart valve, is an “open heart” surgical procedure. Briefly, the procedure necessitates a surgical opening of the thorax, initiation of extra-corporeal circulation with a heart-lung machine, stopping and opening the heart, excision and replacement of the diseased valve, and re-starting of the heart. While valve replacement surgery typically carries a 1-4% mortality risk in otherwise healthy persons, a significantly higher morbidity is associated with the procedure, largely due to the necessity for extra-corporeal circulation. Further, open heart surgery is often poorly tolerated in elderly patients. Thus, if the extra-corporeal component of the procedure could be eliminated, morbidities and cost of valve replacement therapies would be significantly reduced.

While replacement of the aortic valve in a transcatheter manner is the subject of intense investigation, lesser attention has been focused on the mitral valve. This is in part reflective of the greater level of complexity associated with the native mitral valve and thus a greater level of difficulty with regard to inserting and anchoring the replacement prosthesis.

Recent developments in the field have provided devices and methods for mitral valve replacement with reduced invasion and risk to the patient. Such devices may include a prosthetic valve disposed within the native valve annulus and held in place with an anchor seated against an exterior surface of the heart near the ventricular apex, and such anchors must be at least a certain size to seat against the heart with adequate security. Methods of implanting such devices therefore typically require providing an intercostal puncture of significant size to accommodate the anchor. Trauma to the patient increases as a function of the diameter of the puncture. Accordingly, methods and devices for anchoring a prosthetic heart valve that avoid the need for an intercostal puncture would improve patient outcomes. Further, in prosthetic heart valve systems that include a prosthetic heart valve coupled to an anchor via a tethering device, the tether may need to be tensioned help keep the prosthetic heart valve in a desired position while tissue ingrowth occurs. If such a prosthetic heart valve system is delivered in a fully transcatheter fashion (e.g. transseptally), tensioning the tether may pose certain difficulties. Thus, it would be preferable to have systems that facilitate simple tensioning of tethers of prosthetic heart valve systems, particularly for transseptally delivered versions of such systems.

### BRIEF SUMMARY OF THE DISCLOSURE

According to one aspect of the disclosure, a prosthetic heart valve system includes a prosthetic heart valve, an anchor, a tether, and a tensioning member. The prosthetic heart valve has an expandable stent and a prosthetic valve assembly disposed within the stent, the prosthetic valve assembly configured to allow blood to flow in a direction from an inflow end of the stent toward an outflow end of the stent and to substantially block blood from flowing from the outflow end of the stent toward the inflow end of the stent. The anchor is adapted to be disposed on or adjacent an epicardial surface of a heart of a patient. The tether has a first end coupled to the anchor and a second end coupled to the prosthetic heart valve. The tensioning member is disposed along an intermediate portion of the tether between the first end of the tether and the second end of the tether, the tether forming a loop around the tensioning member. The tensioning member is adjustable to increase or decrease a size of the loop to change an effective length of the tether between the prosthetic heart valve and the anchor.

According to another aspect of the invention, a prosthetic heart valve system includes a prosthetic heart valve, an anchor, a tensioning member, and a tether. The prosthetic heart valve has an expandable stent and a prosthetic valve assembly disposed within the stent, the prosthetic valve assembly configured to allow blood to flow in a direction from an inflow end of the stent toward an outflow end of the stent and to substantially block blood from flowing from the outflow end of the stent toward the inflow end of the stent. The anchor is adapted to be disposed on or adjacent an epicardial surface of a heart of a patient. The tensioning member has an externally threaded inner shaft received within an internally threaded outer housing. The tether has a first end coupled to the inner shaft, the inner shaft rotatable relative to the outer housing to: (i) translate the inner shaft into the outer housing to draw the tether closer to the outer housing; or (ii) translate the inner shaft out of the outer housing to move the tether away from the outer housing. In an implanted condition of the prosthetic heart valve system, either (i) the outer housing is fixed to the prosthetic heart valve and a second end of the tether is fixed to the anchor; or (ii) the outer housing is fixed to the anchor and the second end of the tether is fixed to the prosthetic heart valve.

According to another aspect of the disclosure, a prosthetic heart valve system includes a prosthetic heart valve, an anchor, a tether clamp, and a tether. The prosthetic heart valve has an expandable stent and a prosthetic valve assembly disposed within the stent, the prosthetic valve assembly configured to allow blood to flow in a direction from an inflow end of the stent toward an outflow end of the stent and to substantially block blood from flowing from the outflow end of the stent toward the inflow end of the stent. The anchor is adapted to be disposed on or adjacent an epicardial surface of a heart of a patient. The tether clamp has a first clamp portion, a second clamp portion, a base, and a cuff, the tether. The tether has a first end coupled to either the anchor or the prosthetic heart valve, the tether passing through an aperture in the base, between the first and second clamp portions, and through an aperture in the cuff. The first and second clamp portions have an unclamped condition in which clamping faces of the first and second clamp portions are spaced away from each other, and a clamped condition in which the clamping faces contact each other.

### BRIEF DESCRIPTION OF THE DRAWINGS

FIG. 1 is an exploded view of a prosthetic heart valve.

FIG. 2 is an opened and flattened view of an unexpanded inner frame of the prosthetic heart valve of FIG. 1.

FIGS. 3 and 4 are side and bottom views, respectively, of the inner frame of FIG. 2 in an expanded configuration.

FIG. 5 is an opened and flattened view of an unexpanded outer frame of the prosthetic heart valve of FIG. 1.

FIGS. 6 and 7 are side and top views, respectively, of the outer frame of FIG. 5 in an expanded configuration.

FIGS. 8-10 are side, front, and top views, respectively, of an assembly of the inner frame of FIGS. 2-4 and the outer frame of FIGS. 5-7, all in an expanded configuration.

FIG. 11A is a perspective view of an anchor for the prosthetic valve of FIG. 1.

FIG. 11B is an axial view of the anchor of FIG. 11A.

FIG. 12 is a side view of the anchor for the prosthetic valve of FIG. 1 according to another arrangement.

FIG. 13 is a perspective view of the anchor of FIG. 11 in a partially everted state.

FIG. 14 illustrates a trans-jugular insertion of a delivery tube for the anchor of FIG. 11.

FIG. 15 illustrates a trans-femoral insertion of the delivery tube of FIG. 14.

FIG. 16 illustrates the delivery tube of FIGS. 14 and 15 extending through a wall of a heart.

FIGS. 17-20 illustrate the anchor of FIG. 11 in progressive stages of deployment from the delivery tube of FIGS. 14 and 15.

FIGS. 21A and 21B illustrate the delivery tube being retracted from the anchor of FIG. 11.

FIG. 22A is a perspective view of a locking clamp in a locked condition.

FIG. 22B is a top view of a base of the locking clamp of FIG. 22A.

FIGS. 22C-D are top views of first and second clamp portions, respectively, of the locking clamp of FIG. 22A.

FIG. 22E is a top perspective view of a cuff of the locking clamp of FIG. 22A.

FIG. 22F is a perspective view of the locking clamp of FIG. 22A in an unlocked condition.

FIG. 23A is a perspective view of a tensioning device in an extended condition.

FIGS. 23B-C are top and bottom perspective views, respectively, of a sliding component of the tensioning device of FIG. 23A.

FIGS. 23D-E are top and bottom perspective views, respectively, of a base component of the tensioning device of FIG. 23A.

FIG. 23F is a cross-section of the base component of the tensioning device of FIG. 23A taken along a first plane.

FIG. 23G is a cross-section of the base component of the tensioning device of FIG. 23A taken along a second plane orthogonal to the first plane of FIG. 23F.

FIGS. 23H-I are perspective views of the tensioning device of FIG. 23A in contracted and extended conditions, respectively.

FIG. 24A is a side view of a tensioning device according to an embodiment of the disclosure.

FIGS. 24B-C are top perspective and cross-sectional views, respectively, of an outer housing of the tensioning device of FIG. 24A.

FIGS. 24D-E are top perspective and cross-sectional views, respectively, of a shaft member of the tensioning device of FIG. 24A.

FIG. 24F is a cross-section of the tensioning device of FIG. 24A coupled to a tether.

FIG. 25A is a highly schematic side view of a tensioning device according to another embodiment of the disclosure.

FIG. 25B is a highly schematic side view of an outer housing of the tensioning device of FIG. 25A.

FIG. 25C is a highly schematic side view of an inner shaft of the tensioning device of FIG. 25A.

### DETAILED DESCRIPTION

As used herein, the term “proximal,” when used in connection with a delivery device or components of a delivery device, refers to the end of the device closer to the user of the device when the device is being used as intended. On the other hand, the term “distal,” when used in connection with a delivery device or components of a delivery device, refers to the end of the device farther away from the user when the device is being used as intended. Further, the term “inflow end” when used herein in connection with a prosthetic atrioventricular valve refers to the end of the prosthetic valve nearest the atrium when the prosthetic valve is implanted in an intended position and orientation, while the term “outflow end” refers to the end of the prosthetic valve nearest the ventricle when the prosthetic valve is implanted in the intended position and orientation. As used herein, the terms “substantially,” “generally,” “approximately,” and “about” are intended to mean that slight deviations from absolute are included within the scope of the term so modified.

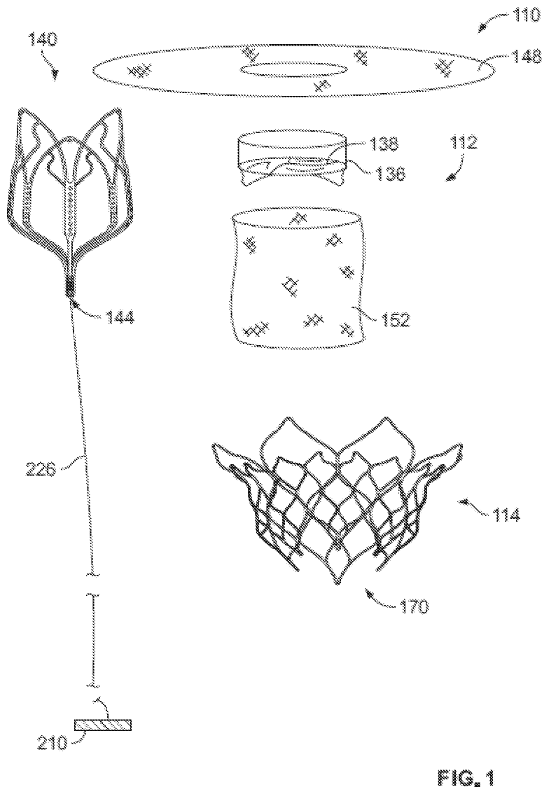

An exemplary prosthetic heart valve 110 as may be used with various embodiments of the present disclosure is shown in an exploded view in FIG. 1. Valve 110 includes an inner structure or assembly 112 and an outer structure or assembly 114. Valve 110 may be coupled to a tether 226 and a collapsible tether anchor 210.

Inner assembly 112 may include an inner frame 140, outer wrap 152 which may be cylindrical, and leaflet structure 136 (including articulating leaflets 138 that define a valve function). Leaflet structure 136 may be sewn to inner frame 140, and may use parts of inner frame 140 for this purpose, although methods of attachment other than sutures may be suitable. Inner assembly 112 is disposed and secured within outer assembly 114, as described in more detail below.

Outer assembly 114 includes outer frame 170. Outer frame 170 may also have in various embodiments an outer frame cover of tissue or fabric (not pictured), or may be left without an outer cover to provide exposed wireframe to facilitate in-growth of tissue. Outer frame 170 may also have an articulating collar or cuff (not pictured) covered by a cover 148 of tissue or fabric.

Tether 226 is connected to valve 110 by inner frame 140. Thus, inner frame 140 includes tether connecting or clamping portion 144 by which inner frame 140, and by extension valve 110, is coupled to tether 226.

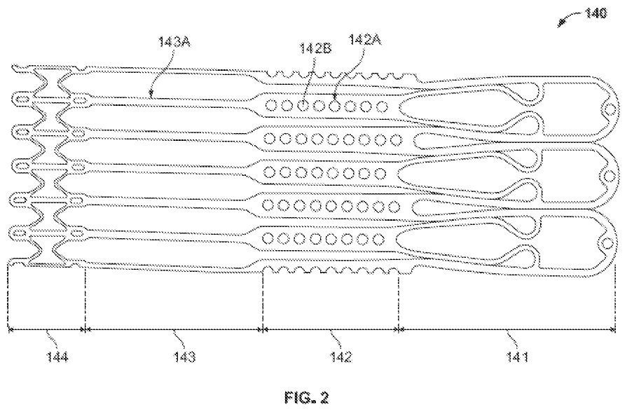

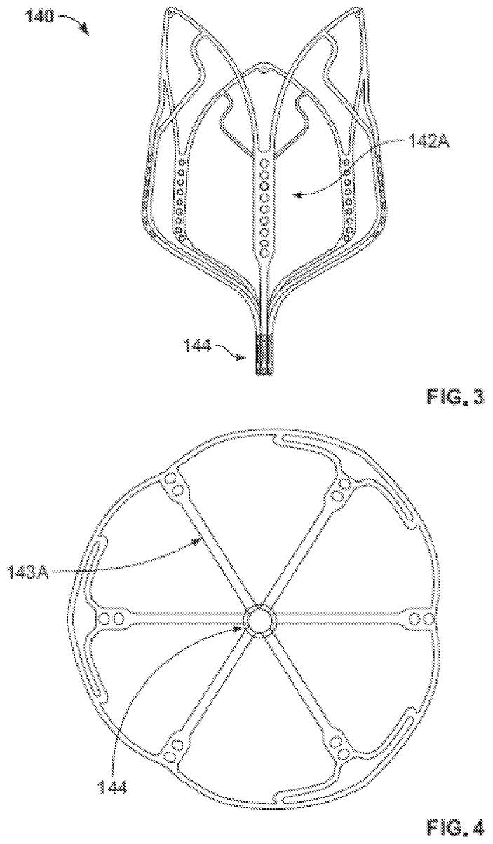

Inner frame 140 is shown in more detail in FIGS. 2-4. Inner frame 140 can be formed from a milled or laser-cut tube of a shape-memory material such as, for example, nitinol. Inner frame 140 is illustrated in FIG. 2 in an initial state, e.g., as milled or laser-cut, but cut longitudinally and unrolled into a flat sheet for ease of illustration. This initial state may generally correspond to a collapsed condition. Inner frame 140 is shown unconstrained, e.g., to the expanded, deployed configuration, in the side view and bottom view of FIGS. 3 and 4, respectively. Inner frame 140 can be divided into four portions corresponding to functionally different portions of inner frame 140 in final form: apex portion 141, body portion 142, strut portion 143, and tether connecting portion 144. Strut portion 143 includes six struts, such as strut 143A, which connect body portion 142 to connecting portion 144. A greater or fewer number of struts 143A is contemplated herein.

Connecting portion 144 includes longitudinal extensions of the struts 143A, connected circumferentially to one another by pairs of v-shaped connecting members, which may be referred to herein as “micro-V's.” Connecting portion 144 is configured to be radially collapsed by application of a compressive force, which causes the micro-V's to become more deeply V-shaped, with each pair of vertices moving closer together longitudinally and the open ends of the V shapes moving closer together circumferentially. When collapsed, connecting portion 144 can clamp or grip one end of tether 226, either connecting directly onto a tether line (e.g., braided filament line) or onto an intermediate structure, such as a polymer or metal piece that is, in turn, firmly fixed to the tether line. The foregoing is merely exemplary and other techniques can be used to connect tether 226 to connecting portion 144, as will be discussed below in further detail.

In contrast to connecting portion 144, apex portion 141 and body portion 142 are configured to be expanded radially. Strut portion 143 forms a longitudinal connection, and radial transition, between the expanded body portion 142 and the compressed connecting portion 144.

Body portion 142 includes six longitudinal posts, such as post 142A, although the body portion may include a greater or fewer number of such posts. The posts 142A can be used to attach leaflet structure 136 to inner frame 140, and/or can be used to attach inner assembly 112 to outer assembly 114, such as by connecting inner frame 140 to outer frame 170. In the illustrated example, posts 142A include apertures 142B through which connecting members (such as suture filaments and/or wires) can be passed to couple the posts to other structures.

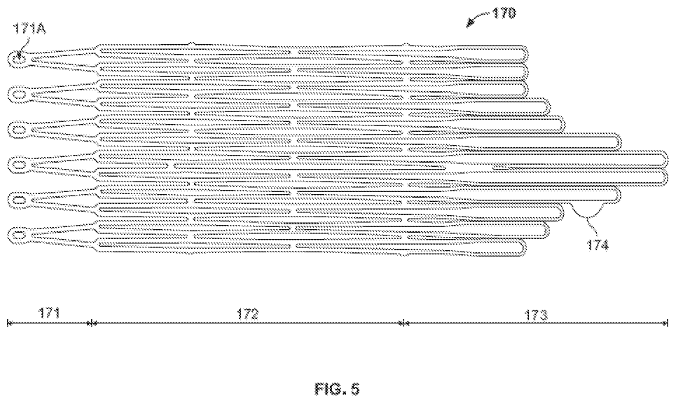

Outer frame 170 of valve 110 is shown in more detail in FIGS. 5-7. Outer frame 170 can be formed from a milled or laser-cut tube of a shape-memory material such as, for example, nitinol. Outer frame 170 is illustrated in FIG. 5 in an initial state, e.g., as milled or laser-cut, but cut longitudinally and unrolled into a flat sheet for ease of illustration. This initial state may generally correspond to the collapsed condition. Outer frame 170 can be divided into a coupling portion 171, a body portion 172, and a flared portion 173, as shown in FIG. 5. Coupling portion 171 may include multiple openings or apertures 171A by which outer frame 170 can be coupled to inner frame 140, as described in greater detail below.

Flared portion 173 may include an indicator 174. In one example, indicator 174 is simply a broader portion of the wire frame element of flared portion 173. Indicator 174 may be more apparent in radiographic or other imaging modalities than the surrounding wireframe elements of flared portion 173. In other examples, indicator 174 can be any distinguishable feature (e.g., protrusion, notch, etc.) and/or indicia (e.g., lines, markings, tic marks, etc.) that enhance the visibility of the part of flared portion 173 on which it is formed, or to which it is attached. Indicator 174 can facilitate the implantation of the prosthetic valve by providing a reference point or landmark that the operator can use to orient and/or position the valve (or any portion of the valve) with respect to the native valve annulus or other heart structure. For example, during implantation, an operator can identify (e.g., using echocardiography) indicator 174 when the valve 110 is situated in a patient's heart. The operator can therefore determine the location and/or orientation of the valve and make adjustments accordingly.

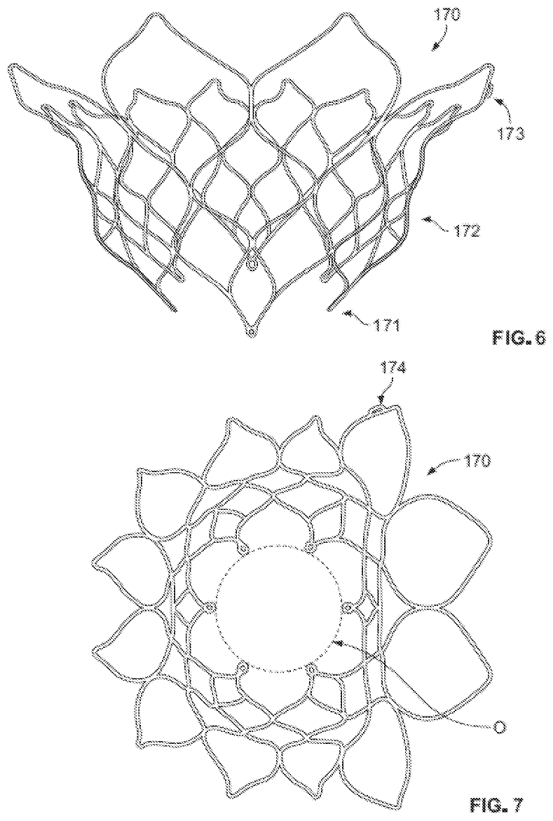

Outer frame 170 is shown in an expanded, deployed configuration, in the side view and top view of FIGS. 6 and 7, respectively. As best seen in FIG. 7, the lower end of coupling portion 171 may form a roughly circular opening (identified by “0” in FIG. 7). The diameter of this opening preferably corresponds approximately to the diameter of body portion 142 of inner frame 140, when the inner frame is in the expanded condition, to facilitate the coupling together of these two components of valve 110.

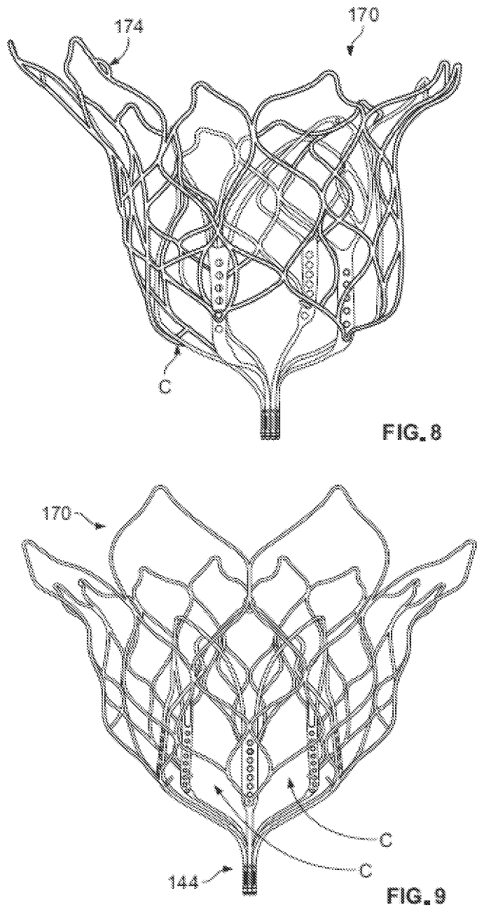

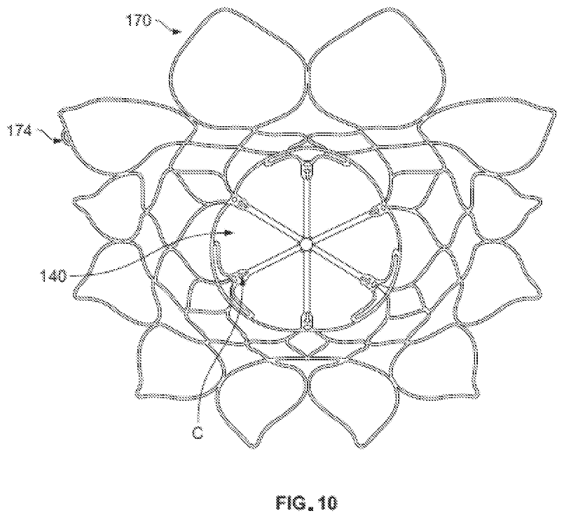

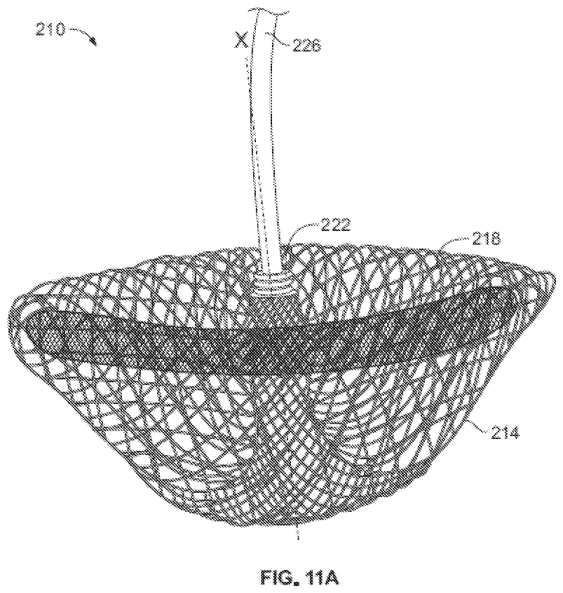

Outer frame 170 and inner frame 140 are shown coupled together in FIGS. 8-10 in front, side, and top views, respectively. The two frames collectively form a structural support for a valve leaflet structure, such as leaflet structure 136 in FIG. 1. The frames support leaflet structure 136 in the desired relationship to the native valve annulus, support the coverings for the two frames to provide a barrier to blood leakage between the atrium and ventricle, and couple to the tether 226 (by the inner frame 140) to aid in holding the prosthetic valve in place in the native valve annulus by the connection of the free end of the tether and tether anchor 210 to the ventricle wall, as described more fully below. The two frames are connected at six coupling points (representative points are identified as “C”). In this embodiment, the coupling of the frames is implemented with a mechanical fastener, such as a short length of wire, passed through an aperture 171A in coupling portion 171 of outer frame 170 and a corresponding aperture 142B in a longitudinal post 142A in body portion 142 of inner frame 140. Inner frame 140 is thus disposed within the outer frame 170 and securely coupled to it.

Prosthetic heart valve 110 may be configured for delivery to a native mitral valve via different delivery routes. For example, prosthetic heart valve 110 may be configured for transapical delivery to the native mitral valve annulus. In a transapical delivery, typically tether 226 is fixed to the prosthetic valve 110 during delivery, and after the prosthetic heart valve 110 is positioned in the native valve annulus, the anchor 210 is passed over a free end of the tether 226. Then, with the anchor 210 positioned against an outside or epicardial surface of the heart, the tether 226 is tensioned to a desired level and the anchor 210 is locked to the tether 226 at the desired tension. However, prosthetic heart valve 110 may also be suited for trans septal delivery to the heart, for example via a trans-femoral or trans-jugular route. In those embodiments it may be preferable for the anchor 210 to be fixed to the tether 226 during delivery, with the opposite end of the tether 226 tensioned and fixed to the prosthetic heart valve 110 after the anchor 210 is deployed. However, as is described in greater detail below, transseptal delivery of prosthetic heart valve 110 may be performed while both ends of the tether 226 are fixed to the anchor 210 and the prosthetic heart valve 110, with tensioning of the tether 226 performed with a separate tensioning mechanism.

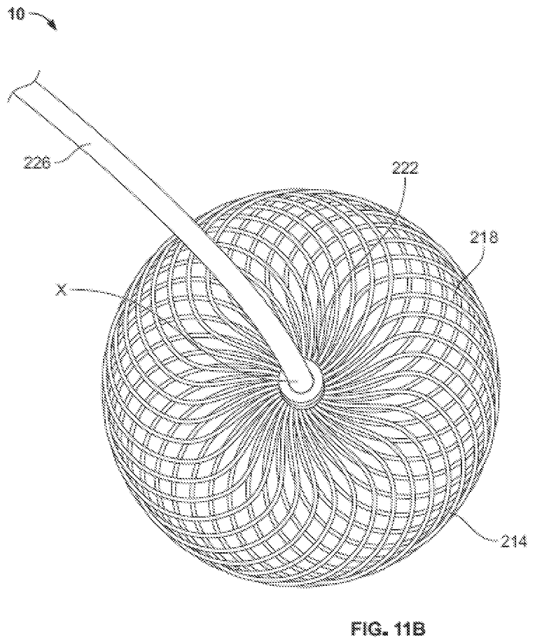

An exemplary anchor 210 for a prosthetic mitral heart valve is illustrated in FIGS. 11A and 11B. The anchor shown in FIGS. 11A-B may be particularly suited for transseptal delivery of a prosthetic mitral valve, although the anchor may be suited for other delivery routes as well. Anchor 210 includes a first disc 214 and a second disc 218, both provided by a wire mesh and centered on an axis X. First disc 214 is offset from second disc 218 in a first direction along axis X. First disc 214 and second disc 218 are each biased toward a dome-shaped resting configuration that is concave toward a second direction along axis X, the second direction being opposite the first direction. The resting configuration of first disc 214 extends far enough in the second direction along axis X to partially overlap second disc 218.

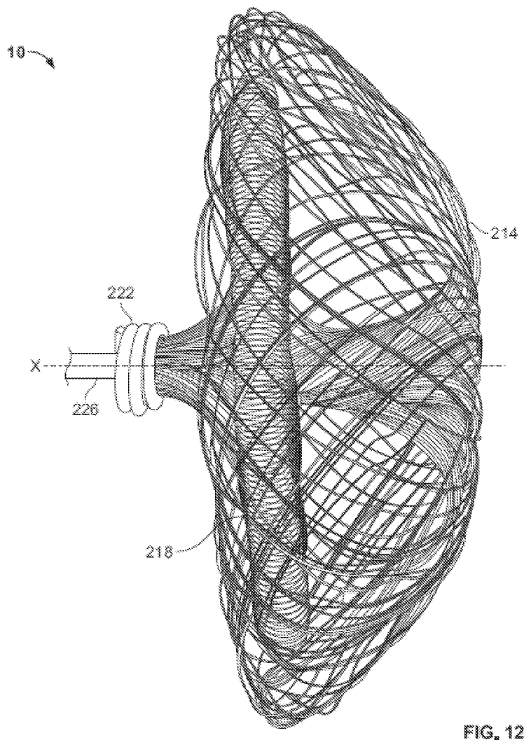

It should be understood that the illustrated dome shapes are merely exemplary, and first disc 214 and second disc 218 may be biased differently. For example, either or both of first disc 214 and second disc 218 may be biased toward a resting configuration that is convex toward the second direction or generally planar. Further, the first disc 214 and second disc 218 may be biased to different resting configurations. In one example, the first disc 214 may be biased toward a dome-shaped resting configuration that is concave toward the second direction while the second disc 218 is biased toward a generally planar configuration having about the same diameter location as the widest part of the dome-shaped resting configuration of the first disc 214, as shown in FIG. 12. In the arrangement shown in FIG. 12, second disc 218 is generally planar in shape with a shallow concavity toward the first direction near the center of second disc 218.

Anchor 210 may also include a cuff 222 for gripping a tether 226, which may be connected to a prosthetic heart valve. Cuff 222 is offset from second disc 218 in the second direction along axis X. One-way gripping features, such as angled teeth, within cuff 222 may permit anchor 210 to slide along tether 226 in the second direction, but not the first direction. In other embodiments, cuff 222 may be fixedly attached to tether 226 so that the anchor 210 may not slide along the tether.

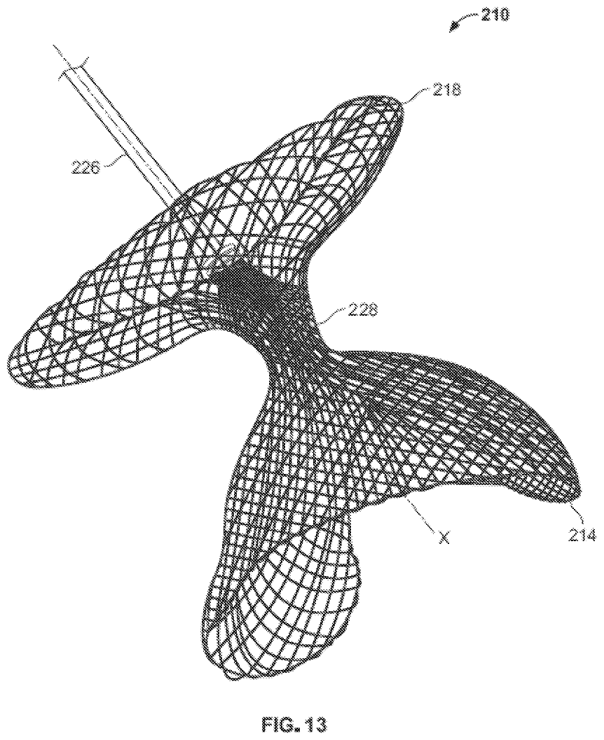

Anchor 210 is flexible, as illustrated in FIG. 13, which shows anchor 210 with the first disc 214 everted from its resting configuration. First disc 214 is connected to second disc 218 by a neck 228 extending between first disc 214 and second disc 218. In the illustrated example, neck 228 is centered on axis X, but in other examples neck 228 may be radially offset from axis X. First disc 214, second disc 218, and neck 228 may all be constructed from a single continuous piece or tube of wire mesh. The wire mesh may be formed from a plurality of strands or wires braided into various three-dimensional shapes and/or geometries to engage tissues, or from one or more sheets cut to provide mesh, such as by laser. In one example, the wires form a braided metal fabric that is resilient, collapsible and capable of heat treatment to substantially set a desired shape. One class of materials which meets these qualifications is shape-memory alloys, such as nitinol. The wires may comprise various materials other than nitinol that have elastic and/or memory properties, such as spring stainless steel, tradenamed alloys such as Elgiloy® and Hastelloy®, CoCrNi alloys (e.g., tradename Phynox), MP35N®, CoCrMo alloys, or a mixture of metal and polymer fibers. Depending on the individual material selected, the strand diameter, number of strands, and pitch may be altered to achieve the desired shape and properties of anchor 210. Shape memory materials such as nitinol may be particularly suitable for anchor 210 in that shape memory material construction enables anchor 210 to consistently return to an intended shape after being compressed and deployed. In other arrangements, anchor 210 may be covered by or may incorporate other flexible biocompatible material, such as a fabric. Although anchor 210 is one example of an expandable anchor that may be suitable for use with the prosthetic valves of the present disclosure, it should be understood that other anchors, including other expandable anchors, may also be suitable.

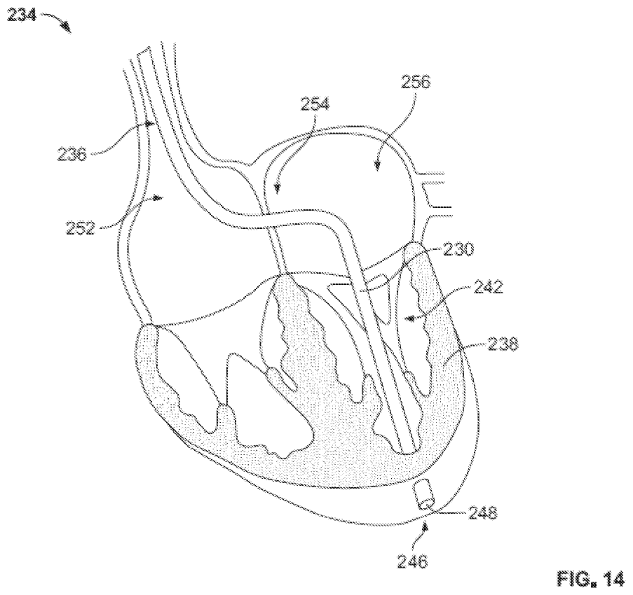

FIG. 14 shows a trans-jugular insertion of an at least partially flexible delivery tube 230 for anchor 210 and valve 110. Delivery tube 230 may be formed of any known material for building catheters, including biocompatible metals such as steel, and may be part of a steerable or flexible catheter system. Delivery tube 230 may include an inflexible portion near its distal end to facilitate the intended puncture of tissue and guidance of valve 110. Delivery tube 230 is inserted through the patient's jugular vein (not shown), then through superior vena cava 236, right atrium 252, atrial septum 254, left atrium 256, native mitral valve 260, and into left ventricle 242. Tube 230 exits left ventricle 242 through ventricular wall 238 at or near the apex 246 of heart 234. A retractable puncturing device (not shown) and a retractable atraumatic tip (not shown) may extend from the distal open end 248 of tube 230 in alternate stages of insertion of tube 230. The puncturing device may produce openings through atrial septum 254 and ventricular wall 238 while the atraumatic tip may act to prevent injury to other tissue. Once delivery tube 230 has been fully inserted, the distal open end 248 of tube 230 is positioned outside of ventricular wall 238. The trans-jugular insertion of tube 230 may be accomplished by any of variety of methods, such as, for example, guiding tube 230 along a guide wire, such as a shape-memory guide wire, inserted through the jugular vein. The flexible nature of anchor 210 allows trans-jugular delivery of anchor 210 through tube 230. Because tube 230, anchor 210, and valve 110 all reach heart 234 from the jugular vein, valve 110 and anchor 210 may be delivered and implanted without any intercostal puncture.

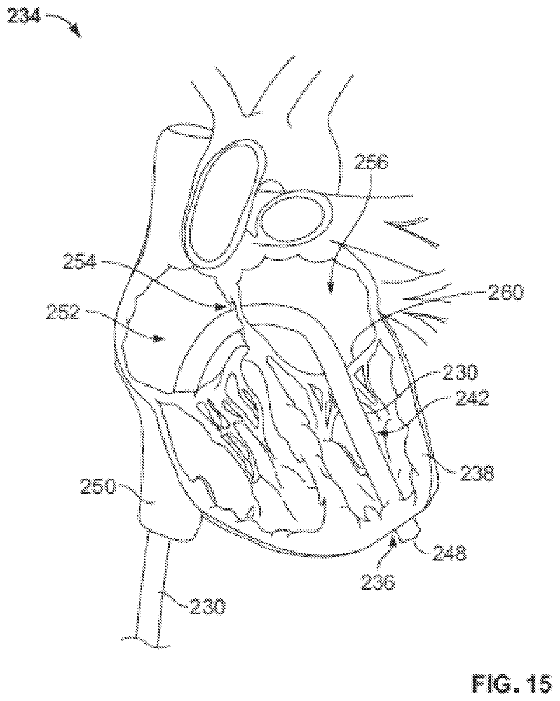

FIG. 15 shows a trans-femoral insertion of tube 230. Tube 230 enters heart 234 through inferior vena cava 250, travels through right atrium 252, and punctures septum 254 to enter left atrium 256. Tube 230 is advanced from left atrium 256 through native mitral valve 260, left ventricle 242, and ventricular wall 238 such that the open end 248 of the tube is positioned outside of wall 238 at or near apex 246. As with trans-jugular insertion, guidance of tube 230 during trans-femoral insertion may be accomplished using a variety of methods, including guidance along a guide wire.

The trans-jugular and trans-femoral insertions described above are merely exemplary. It should be understood that tube 230 could be guided toward heart 234 using any suitable method known in the art.

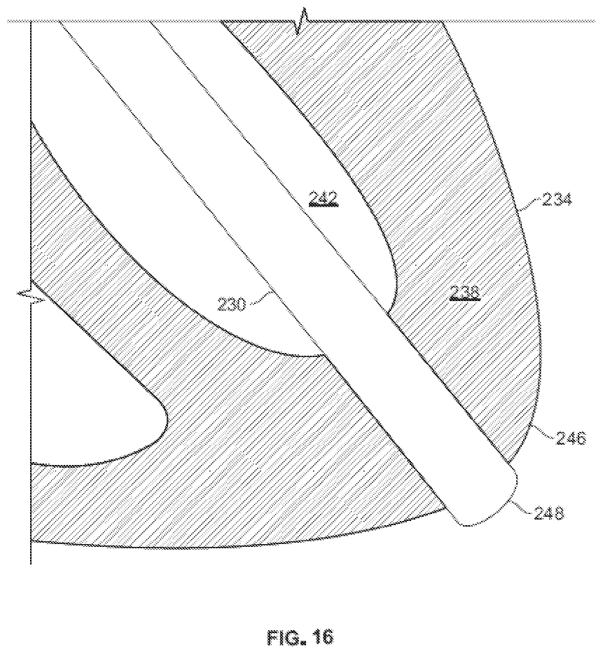

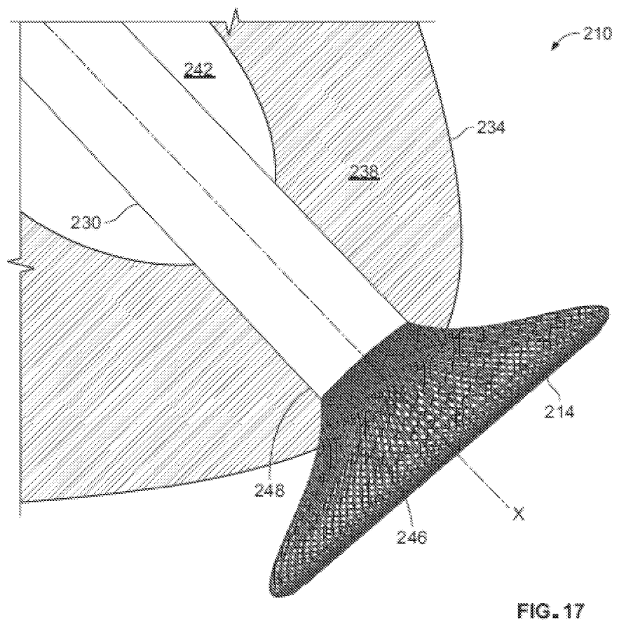

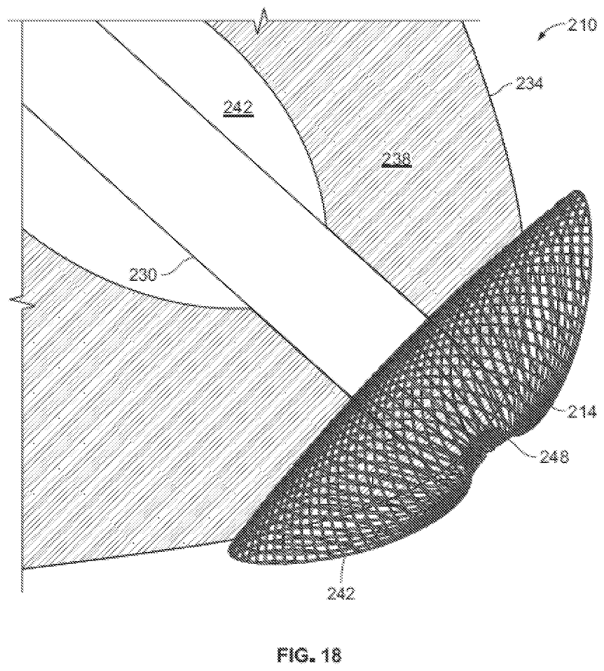

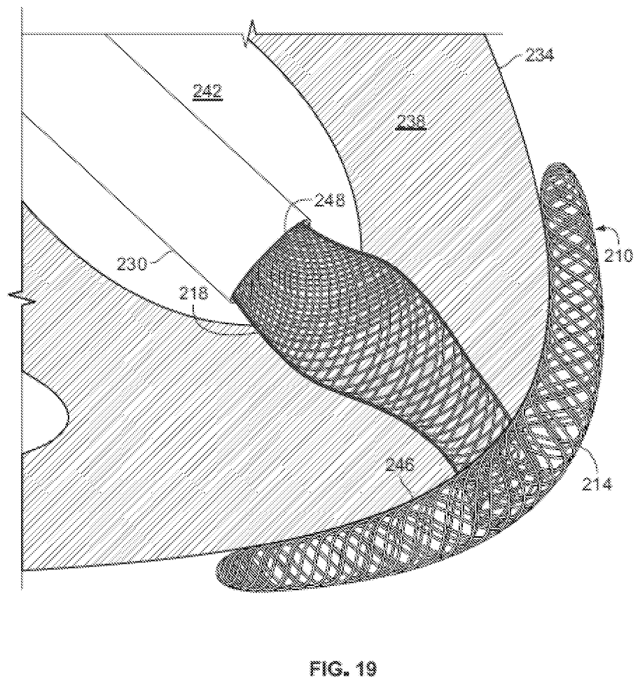

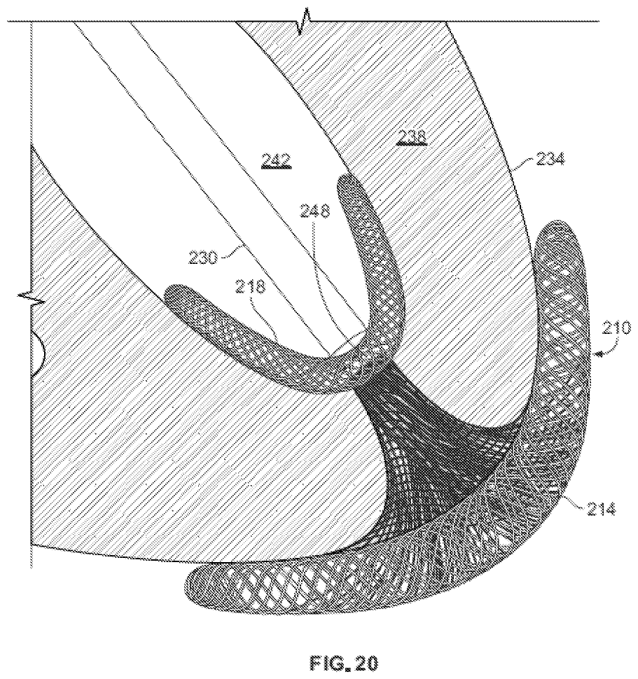

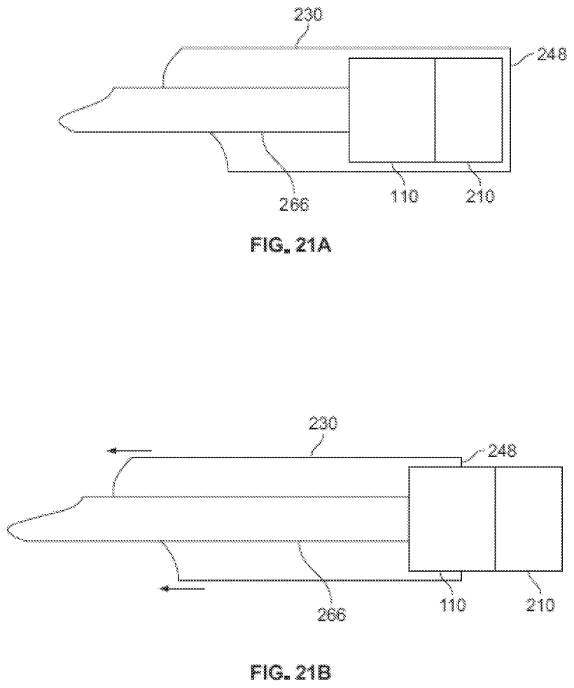

FIGS. 16-20 illustrate anchor 210 in progressive stages of deployment from the open end 248 of tube 230. Tube 230 is shown in a distalmost position in FIG. 16, with open end 248 positioned outside of heart 234. Tube 230 may be retracted while anchor 210 is forced to remain in place, such as by a reversal of a typical Bowden cable arrangement. For example, a semi-rigid cable or wire 266 may be inserted through tube 230 to contact the proximal end of anchor 210, as shown in FIG. 21A. Pulling tube 230 proximally relative to wire 266 causes anchor 210 to deploy out from the open end 248 of tube 230, as shown in 21B. As shown in FIG. 17, retracting tube 230 while preventing anchor 210 from retreating with the tube into heart 234 causes first disc 214 of anchor 210 to deploy out from the open end 248 of tube 230 and expand radially relative to axis X. Upon further retraction of tube 230, the bias of first disc 214 causes it to curve back onto the outer apex 246 of heart 234, as shown in FIG. 18. Further retraction of tube 230 in FIG. 19 allows second disc 218 to deploy and expand radially relative to axis X within left ventricle 242 until second disc 218 opens to press against an inner side of wall 238, as shown in FIG. 20. Pressure against wall 238 results from the elastic bias of first disc 214 and second disc 218 toward certain resting positions as described above with regard to FIGS. 11A, 11B, and 12. First disc 214 and second disc 218 pressing on opposite sides of wall 238 causes anchor 210 to grip wall 238. Such progressive expansion from within a narrow tube results in anchor 210 adequately securing valve 110 to ventricular wall 238 without requiring an intercostal puncture through the patient's chest.

In the trans-jugular and trans-femoral delivery routes described above, one end of the tether 226 is preferably fixed to the anchor 210 before deployment of the anchor 210, including prior to loading the valve 110 into tube 230. Tether 226 and anchor 210 remain attached while anchor 210 is delivered to the exterior of ventricular wall 238 from within tube 230, and tether 226 is uncovered by the retraction of tube 230. In alternate embodiments, the anchor 210 and tether 226 may be fixed to one another during or immediately after deployment of the anchor 210. The tether 226 may extend proximally to a second free end, and the prosthetic valve 110 is preferably delivered over the tether 226, using the tether 226 as a rail and/or guide, with the tether 226 extending through a center portion of prosthetic valve 110 while the valve 110 is being delivered to the native valve annulus. Once the prosthetic valve 110 is at or adjacent the final position within the native valve annulus, the prosthetic valve 110 is preferably fixed to the tether 226 by engagement or activation of the tether connecting portion 144 with the tether 226. This engagement may be accomplished via one or more mechanisms, including those described below, and the engagement may occur just before, during, or just after deployment of the prosthetic valve 110. Much of the below disclosure relates to mechanisms to facilitate such a connection between the prosthetic valve 110 and the second end of the tether 226 and/or to facilitate tensioning of the tether 226 while the tether 226 is fixed to both the prosthetic valve 110 and the anchor 210. Placement of valve 110 into native mitral valve 260 may involve affixing tether 226 to anchor 210 at one end of tether 226, as noted above. It may also involve affixing tether 226 to a tensioning mechanism (not shown) at the other end of tether 226. The tensioning mechanism may include a load sensor for measuring tension and will remain outside the body of the patient while anchor 210 is inserted through one of the above-mentioned methods. Although it should be understood that the tensioning mechanism may be affixed to the tether after the anchor 210 is deployed. Thus, after anchor 210 is secured against outer apex 246 of heart 234, tether 226 may extend from anchor 210 to the tensioning mechanism, e.g., the entire length of the path used to insert anchor 210 against outer apex 246. One example using an above-mentioned method may embody tether 226 extending from anchor 210 through ventricular wall 238, left ventricle 242, native mitral valve 260, left atrium 256, atrial septum 254, right atrium 252, superior vena cava 236, exiting the patient through a jugular vein (not shown) and attaching to the tensioning mechanism outside the patient. Maintenance of tether 226 in this position may permit valve 110 to attach to tether 226 outside the patient's body, using tether 226 as a guide to follow the path of tether 226 to place valve 110 within native mitral valve 260. Several embodiments of how valve 110 may be secured into place in native mitral valve 260 with the desired tether tension level will be explained below in further detail. It should be understood that, if the prosthetic valve 110 is fixed to the tether 226 after the anchor 210 is deployed and the prosthetic valve 110 is at or near its final desired position, it may be preferable to fix the prosthetic valve 110 to the tether 226 by activating or engaging the tether connecting portion 144 to the tether 226 after the tether 226 has been tensioned a desired amount, at which point the coupling of the tether connecting portion 144 of the prosthetic valve 110 to the tether 226 will maintain the desired tension in the tether 226. Such tension may provide certain benefits, for example helping to prevent the prosthetic valve 110 from migrating into the atrium. The force on the ventricle from the tension of the tether 226 may also facilitate more efficient functioning of the ventricle. Various mechanisms for tensioning the tether 226 to a desired level and/or fixing the prosthetic valve 110 to the tether 226 are described in greater detail below.

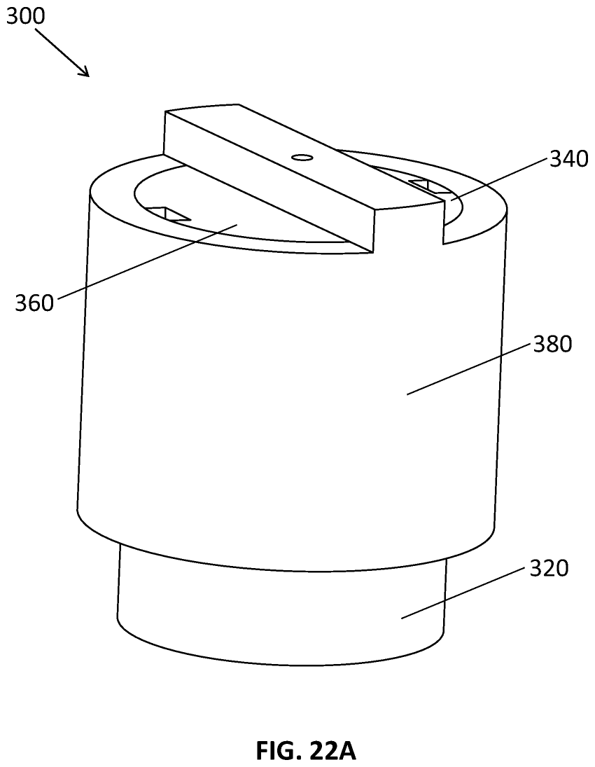

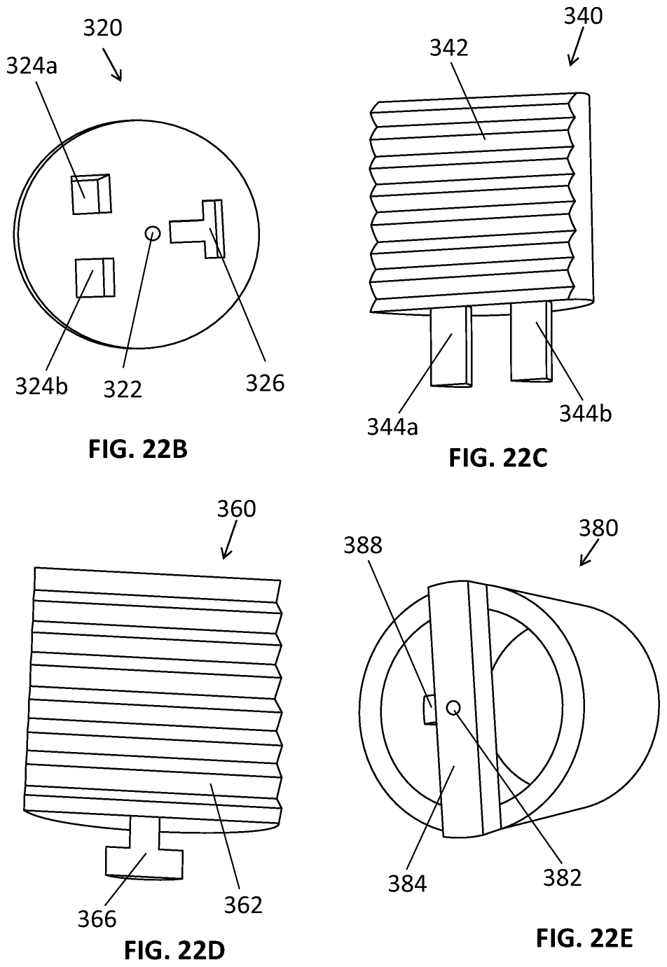

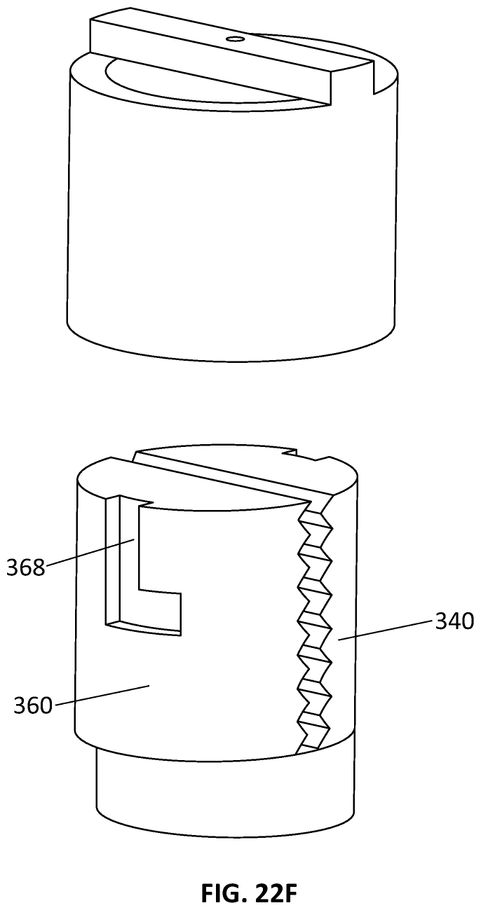

FIG. 22A is a perspective view of a tether locking clamp 300 in an assembled or locked condition according to one aspect of the disclosure. Locking clamp 300 may be particularly well suited for locking tether 226 after tensioning the tether during a transseptal procedure in which anchor 210 and tether 226 are fixed together during delivery, and the tether 226 is only fixed and tensioned to the prosthetic heart valve 110 after the anchor 210 is deployed. However, it should be understood that locking clamp 300 may also be suitable for locking a tether at a desired tension using other delivery routes, such as transapical delivery.

Generally, locking clamp 300 may include a base 320, a first clamp portion 340, a second clamp portion 360, and a cuff 380. Broadly speaking, the tether 226 may be threaded through an aperture 322 in the base 320 and an aperture 382 in the cuff 380, with the two clamping portions 340, 360 configured to come together to clamp the tether 226 when the tether is at the desired tension. FIG. 22B is a top view of base 320, illustrating the surface of the base that interacts with the two clamp portions 340, 360. Generally, the base 320 is cylindrical and includes a central aperture 322 extending along the length of the base 320 between the top face and bottom face (not shown in FIG. 22B) to allow tether 226 to pass through the base 320. On a first side of the aperture 322, the base 320 may include one or more receivers 324a-b adapted to fixedly receive complementary protrusions 344a-b of the first clamp portion 340. In the illustrated embodiment, the base 320 includes two receivers 324a-b, each receiver defining a generally rectangular recess. On the opposite side of the aperture 322, the base 320 may include another receiver 326 adapted to movably receive a complementary protrusion 366 of the second clamp portion 360. In the illustrated example, receiver 326 is substantially “T”-shaped having a first wider rectangular recess farther away from the aperture 322 in communication with a second narrower rectangular recess nearer the aperture 322.

An interior surface of first clamp portion 340 is illustrated in FIG. 22C and an interior surface of second clamp portion 360 is illustrated in FIG. 22D. The interior surfaces of the first and second clamp portions 340, 360 may include complementary teeth 342, 362. In the illustrated embodiment, teeth 342, 362 include generally horizontally extending peaks separated by generally horizontally extending troughs so that, when the interior surfaces of the first and second clamp portions 340, 360 engage each other, the peaks of the teeth 342 of first clamp portion 340 are received within the troughs of the teeth 362 of the second clamp portion 360. As described in greater detail below, when the inner surfaces of clamp portions 340, 360 engage one another with a tether 226 therebetween, the teeth 342, 362 help maintain the tether 226 at a desired tension. Although one particular format for teeth 342, 362 are illustrated, it should be understood that other complementary or engaging friction features may instead be used to achieve the same goal. The first and second clamp portions 340, 360 may each have the shape of half of a right cylinder (best shown in FIG. 22F), so that when the first and second clamp portions engage each other, they form a cylindrical assembly with an outer diameter sized to be received within the cuff 380.

Referring now to FIGS. 22B-C, the first clamp portion 340 may include two protrusions 344a-b extending from a bottom surface of the first clamp portion. Each protrusion 344a-b may be generally rectangular in cross-section to matingly fit within receivers 324a-b to fix the position of the first clamp portion 340 relative to the base 320 when the first clamp portion 340 is assembled to the base 320. However, it should be understood that other shapes and numbers of protrusions 344a-b and receivers 324a-b may be suitable, preferably as long as the first clamp portion 340 remains laterally fixed relative to the base 320 when the first clamp portion 340 is assembled to the base 320. In some embodiments, the base 320 and the first clamp portion 340 may be formed as an integral member, eliminating the need for the protrusions 344a-b and receivers 342a-b.

Referring now to FIGS. 22B and 22D, the second clamp portion 360 may include a single protrusion 366 extending from a bottom surface thereof. Protrusion 366 may be generally “T”-shaped with a first narrow portion extending axially from the bottom surface of the second clamp portion 360, and a second wide portion positioned at the end of the narrow portion to form the “T”-shape. In use, the wide portion of the protrusion 366 may be inserted into the corresponding wide portion of the receiver 326. Then, the second clamp portion 360 may be moved laterally toward the aperture 322 of base 320 with the narrow portion of the protrusion 366 sliding along the narrow portion of the receiver 326. After the second clamp portion 360 is slid laterally toward the center of the base 320, the wide portion of the protrusion 366 is aligned with the narrow portion of the receiver 326. Because the wide portion of the protrusion 366 is larger than the narrow portion of the receiver 326, the protrusion 366 (and thus the second clamp portion 360) is prevented from being pulled out of the receiver 326 of the base 320 after the first clamp portion 360 is slid laterally toward the center of the base 320. Although the specific embodiment illustrates a complementary “T”-shaped protrusion 366 and receiver 326, other shapes may be suitable that allow for lateral movement of the second clamp portion 360 toward and away from the center of the base 320, preferably as long as the second clamp portion 360 is unable from pulling away from the base 320 when it is in the position nearer the center of the base 320. For example, the wide part of the “T”-shaped protrusion 366 may be longer than the corresponding wide part of the “T”-shaped receiver 326 to prevent the second clamp portion 360 from pulling away from, and decoupling from, the base 320.

Although not visible in FIGS. 22C-D, the outer half-cylindrical surface of each clamp portion 340, 360 may include an “L”-shaped groove having a first axial groove section extending from a top surface of the clamp portion 340, 360 that connects with a second circumferential groove section extending along a portion of the outer circumference of the clamp portion. FIG. 22F illustrates the “L”-shaped groove 368 of the second clamp portion 360. The top of each “L”-shaped groove may extend into the top surface of the clamping portion to allow access to the groove from the top surface as best shown in FIG. 22A. In the assembled condition of the two clamp portions 340, 360, the second circumferential groove section of the “L”-shaped groove 368 of the second clamp portion 360 may extend in an opposite direction than the second circumferential groove section of the “L”-shaped groove (not illustrated) of the first clamp portion 340. As described in greater detail below, this configuration allows interiorly extending projections 388 of the cuff 380 to be slid axially over the assembled clamp portions 340, 360 along the first axial groove portions, and then rotated so that the interiorly extending projections 388 traverse the second circumferential groove sections of the “L”-shaped grooves, locking the cuff 380 to the assembled clamp portions 340, 360.

Referring now to FIG. 22E, the cuff 380 may be a hollow cylinder having an inner diameter that is about equal to, or slightly larger than, the outer diameter of the two clamp portions 340, 360 when the clamp portions are assembled to one another with their teeth 342, 362 in engagement with each other. A central bar 384 may extend across a top surface of the cuff 380 and may include a central aperture 382 that axially aligns with the aperture 322 of the base 320 when locking clamp 300 is in use. Described in greater detail below, apertures 322 and 382 may be sized to receive the tether 226 attached to anchor 210. The cuff 380 may also include two interiorly extending projections 388 on diametrically opposed sizes of the inner surface of the cuff 380 (only one projection 388 visible in FIG. 22E). The projections 388 may be sized and shaped to fit within the “L”-shaped grooves 368 of the first and second clamp portions 340, 360.

In one exemplary use, the base 320 may be fixed to the tether connecting portion 144 of the inner frame 140, and the first and second clamping portions 340, 360 may be coupled to the base 320. A collapsible anchor 210 may be delivered first in a transseptal procedure, with one end of the tether 226 fixed to the anchor. After the anchor is positioned against the wall of the heart (for example as shown in FIG. 20), the opposite free end of the tether may first be threaded through aperture 382 of cuff 380, and then through aperture 322 of base 320. The prosthetic heart valve may be delivered over the tether 226 using the tether as a rail, until the prosthetic heart valve is positioned within the native valve annulus. The free end of the tether 226 may be tensioned to a desired tension level, and then the cuff may be pushed or pulled over the first and second clamping portions 340, 360 in order to clamp the tether 226 at the desired tension level, and the cuff 380 may be rotated to lock the cuff relative to the first and second clamping portions 340, 360. The tether 226 will thus be maintained at the desired tension level, and any excess length of the tether may be cut, for example using a cautery device.

Any suitable tool may be used to grip the cuff 380 so that it can be positioned over the first and second clamping portions 340, 360, and then rotated relative to those two clamping portions. However, other alternative designs may help facilitate the clamping and locking action of tether locking clamp 300. For example, in one embodiment, the outer surfaces of the first and second clamping portions 340, 360 may be tapered in a direction away from the base 320, while the interior of cuff 380 may have a corresponding taper. In that embodiment, the cuff 380 may be coupled to the first and second clamping portions 340, 360 so that the cuff cannot axially separate from the first and second clamping portions. However, when the cuff 380 is only minimally overlapping the first and second clamping portions 340, 360, the first and second clamping portions may be spaced apart from one another due to the tapering. As the cuff 380 is pulled (or pushed) toward base 320, the interaction of the tapers will force the first and second clamping portions 340, 360 toward one another to clamp the tether 226 therebetween. In other embodiments, the outer cuff 380 may be axially fixed to the first and second clamping portions 340, 360 so that the entirety of the first and second clamping portions are positioned within the cuff. However, the cuff 380 may include an internal camming mechanism. In that embodiment, when the cuff 380 is in a first rotational position relative to the first and second clamping portions 340, 360, the first and second clamping portions may be spaced apart from each other. Upon rotating the cuff 380 to a second rotational position relative to the first and second clamping portions 340, 360, the clamping portions are forced together to secure the tether 226 therebetween. Any suitable camming mechanism may provide for this functionality. In one example, the cuff 380 may have a generally oblong or elliptical interior profile. When the longer end of the interior oblong shape of the cuff 380 is perpendicular to the clamping faces of the first and second clamping portions 340, 360, the first and second clamping portions can move relative to one another because of the increased space of the oblong interior shape of the cuff. As the cuff 380 is rotated and the interior oblong shape of the cuff 380 becomes parallel to the clamping faces of the first and second clamping portions 340, 360, the first and second clamping portions are forced to move toward each other because of the decreasing available space within the cuff. However, other camming mechanisms may achieve a similar result.

Although the exemplary use of tether locking clamp 300 is described above for a transseptal delivery, it should be understood that the tether locking clamp may similarly be used for a transapical delivery. In a transapical delivery, one end of the tether 226 may be fixedly coupled to the tether connecting portion 144 of the inner frame 140, and the base 320 may be coupled to the epicardial anchor device. Other than these differences, the procedure of using the tether locking clamp 300 to lock the tether at a desired tension may be substantially similar to the transseptal example provided above.

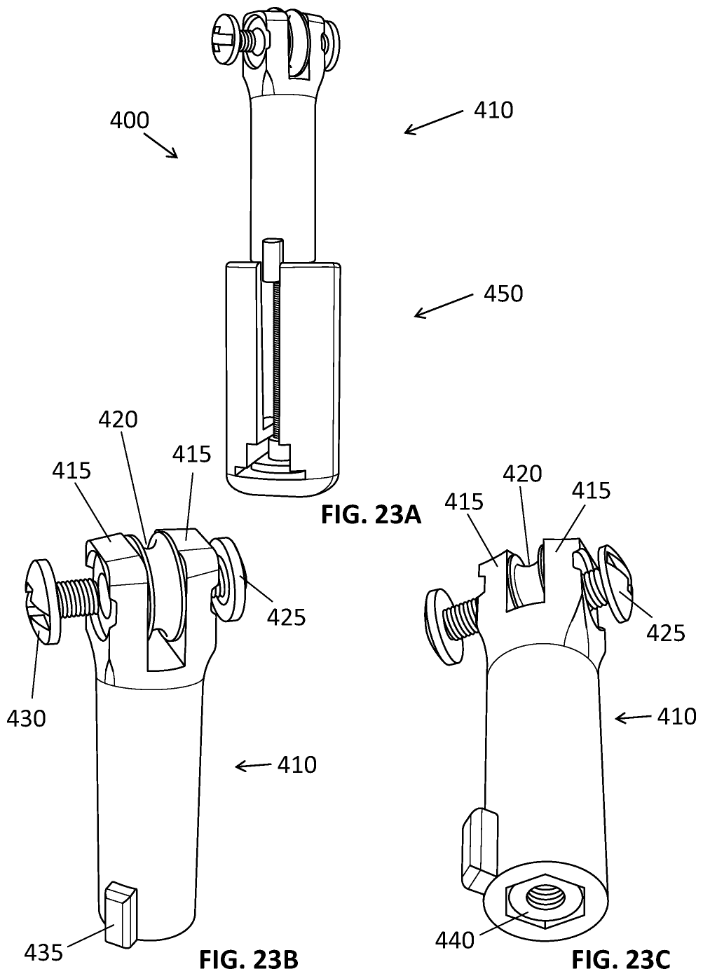

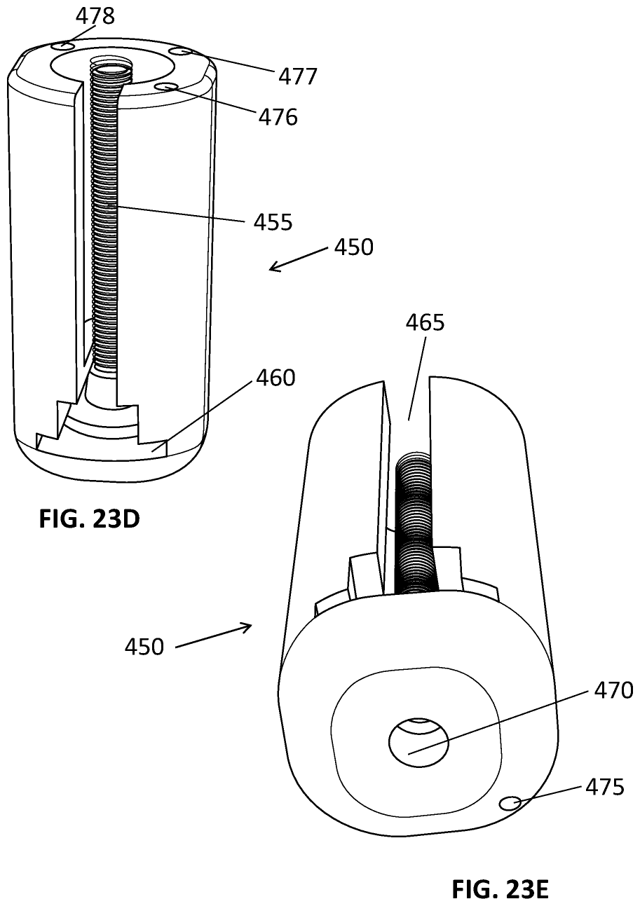

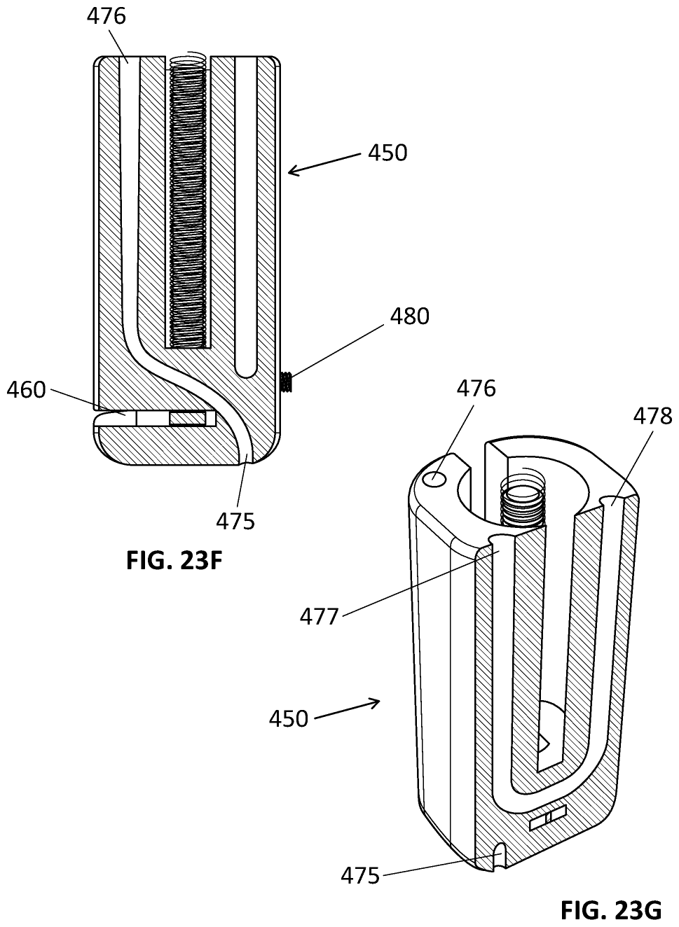

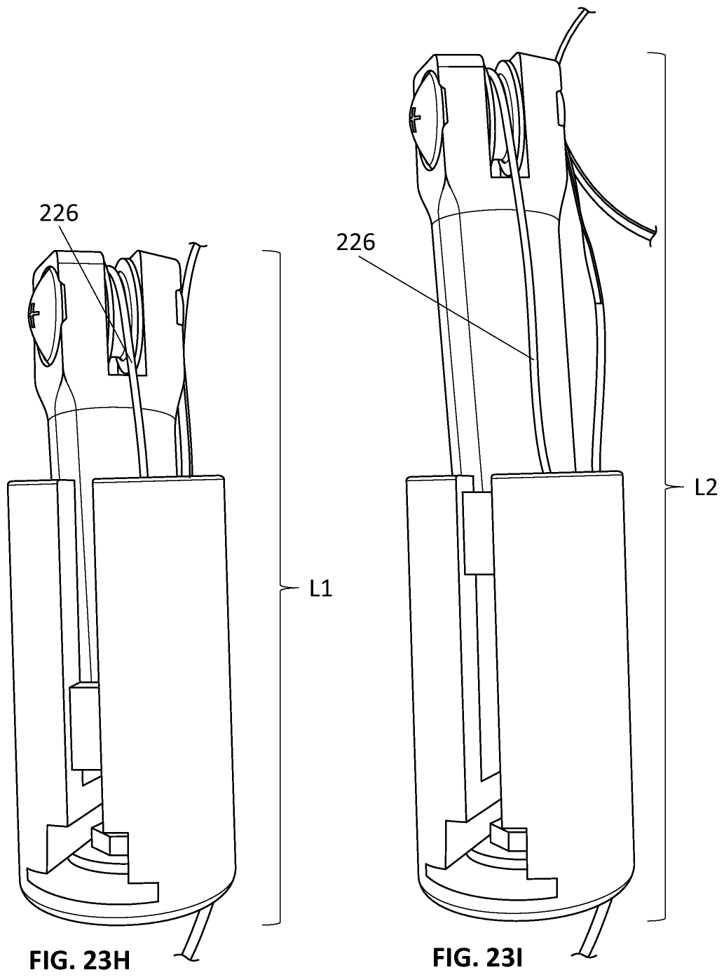

FIG. 23A is a perspective view of a tensioning member 400 that may be used to facilitate tensioning of tether 226. Tensioning member 400 may be used with prosthetic heart valve 110 and anchor 210 in a manner which allows the tether 226 to be fixed to the prosthetic valve 110 at a first end and the anchor 210 at an opposite end for the entire delivery, with the tensioning member 400 coupled to the tether 226 at a point between the prosthetic heart valve 110 and the anchor 210. Although tensioning member 400 may be particularly well suited for fully transcatheter delivery of prosthetic heart valve 110, it should be understood that tensioning member 400 may be used with prosthetic heart valve 110 and anchor 210 with any delivery route. Broadly, the tether 226 may loop around two components of the tensioning member 400 that are capable of telescoping relative to each other, thus increasing or decreasing the tension on the tether 226 during the telescoping motion.

Still referring to FIG. 23A, tensioning member 400 may include a first sliding component 410 and a second base component 450. The first sliding component 410 is illustrated isolated from the second base component 450 in FIGS. 23B-C. First sliding component 410 may be generally cylindrical with a substantially hollow interior for receiving a lead screw 455, with two prongs 415 positioned at a free end of the first sliding component 410. The two prongs 415 may define a gap therebetween adapted to receive a guiding member or pulley 420. The pulley 420 may be rotatable, about its central axis, relative to the prongs 415. For example, a barrel nut 425 having a smooth outer diameter and threaded interior may pass through an aperture in one of the prongs 415 and through a center opening of pulley 420. Screw 430 may pass through an aperture in the other one of the prongs 415 and screw into barrel nut 425 to secure the pulley 420 relative to the prongs 415 while still allowing rotation of the pulley 420. It should be understood that, in FIGS. 23A-C, the barrel nut 425 and screw 430 are shown at least partially spaced away from the prongs 415 in order to better illustrate those components. The pulley 420 may be sized and shaped to allow the tether 226 to wrap around the pulley while generally securing the tether 226 within a groove of the pulley 420. First sliding component 410 may include a short protrusion 435 at an end opposite the prongs 415. In the illustrated embodiment, protrusion 435 may be a generally rectangular, being sized and shaped to be received within a guiding slot 465 of the second base component 450, as described in greater detail below. Still further, a bottom end of first sliding component 410 may include a nut 440. Nut 440 may be generally hexagonal and may be fixed within the first sliding component 410 to prevent rotation of the nut 440 with respect to the first sliding component 410. In one example, the first sliding component 410 may include a complementary hexagonal recess to prevent rotation of the nut 440, although other complementary shapes may be suitable. Additionally or alternatively, the nut 440 may be fixed to the first sliding component 410, for example via adhesives or the like.

The second base component 450 is illustrated isolated from the first sliding component 410 in FIGS. 23D-E. Generally, the second base component 450 may include a generally cylindrical main body that defines a generally cylindrical interior space to receive the first sliding component 410 therein. The second sliding component 450 may include a lead screw 455 axially fixed therein, for example via a head of the lead screw 455 being received within a bottom slot 460 of the second base component 450, the bottom slot 460 being sized to axially trap the head of the lead screw 455, while allowing rotation of the lead screw 455. Lead screw 455 may be assembled to the second base component 450 by sliding the head of the lead screw 455 into bottom slot 460, and the threaded shaft of the lead screw 455 through axially extending guiding slot 465. An aperture 470 may be provided through a bottom surface of the second base component 450, the aperture 470 aligning with the head of the lead screw 455 when the lead screw 455 is assembled to the second base component 450. A driving portion of the head of the lead screw 455, such as a hexagonal recess or other recess configured to engage the head of a driver such as a screwdriver, may be accessible through the aperture 470 to rotate the lead screw 455. When the free end of the lead screw 455 is received through nut 440, the lead screw 455 may be rotated to cause rotation of the lead screw 455 relative to the nut 440. In the assembled condition, shown in FIG. 23A, the short protrusion 435 is received within guiding slot 465, preventing relative rotation between the first sliding component 410 and the second base component 450. As a result, as the lead screw 455 is rotated, the first sliding component 410 will telescope into or out of the second base component 450, depending on the direction of rotation. It should be understood that the tensioning member 400 may include features that prevent complete detachment between the first sliding component 410 and the second base component 450. For example, components may be provided on the lead screw 455 and/or the nut 440 to prevent complete disengagement.

The tensioning member 400 may include various features to guide the tether 226 along the tensioning member 400 between the anchor 210 and the prosthetic heart valve. A first point of exit of the tether 226 may be first exit aperture 475 in the second base component 450 illustrated in FIG. 23E. As shown in the cross-section of FIG. 23F, first exit aperture 475 may be in communication with aperture 476 via a channel extending therebetween, the channel generally extending from the rear of the second base component 450 to the front of the second base component 450. The channel is preferably completely enclosed between the opposing apertures 475, 476. The tether 226 may couple to either the anchor 210 or the prosthetic heart valve 110 and extend into aperture 475, through the channel, and out of aperture 476. From aperture 476, the tether 226 may extend upward and wrap or loop around pulley 420. After wrapping or looping around pulley 420, the tether 226 may extend back downward into aperture 477 of second base member 450, shown best in FIG. 23D. As shown in the cross-section of FIG. 23G, aperture 477 may connect to second exit aperture 478 via a generally “U”-shaped channel that is fully enclosed by the second base component 450 between the apertures 477, 478. The tether 226 may exit from the second exit aperture 478 and then connect to the anchor 210 (if the opposite end of the tether 226 is coupled to the prosthetic heart valve 110), or to the prosthetic heart valve 110 (if the opposite end of the tether 226 is coupled to the anchor 210). As shown in FIG. 23F, a set screw 480 may be provided on the rear of the second sliding component 450, the set screw 480 being capable of being driven into the bottom of the “U”-shaped channel that connects apertures 477, 478. This set screw 480 may help ensure that the tensioning component 400 does not slide relative to the tether 226, particularly during extension and/or contraction of the tensioning component 400, as described below.

FIGS. 23H-I illustrate the tensioning member 400 in a contracted and expanded condition, respectively, with the tether 226 coupled to the tensioning member 400 as described above. In the contracted condition of FIG. 23H, the lead screw 455 is threaded a relatively large distance relative to nut 440 so that a relatively large portion of the first sliding component 410 is received within the second base component 450. As a result, the axial length of tensioning member 400 is a relatively small length L1 in the contracted condition. In the expanded condition of FIG. 23I, the lead screw 455 is threaded a relatively small distance relative to nut 440 so that a relatively small portion of the first sliding component 410 is received within the second base component 450. As a result, the axial length of tensioning member 400 is a relatively large length L2 in the expanded condition relative to the contracted condition. As should be understood from the description above, the tensioning member 400 may be reversibly transitioned between the contracted and extended conditions by rotating the lead screw 455 in the desired direction. Although two discrete positions of the tensioning member 400 are illustrated in FIGS. 23H-I, it should be understood that the axial length of the tensioning member 400 may be adjusted in infinitesimally small increments between the maximum length L2 and minimum length L1 by adjusting the amount of rotation of lead screw 455. Further, it should be understood that the interaction between the exterior threads of the lead screw 455 and the interior threads of the nut 440 will lock the axial position of first sliding component 410 relative to the second base component 450 in the absence of further rotation of the lead screw 455. Because the opposite ends of the tether 226 are fixed to the prosthetic heart valve 110 and the anchor 210, adjusting the length of the tensioning member 400 will increase or decrease tension on the tether 226 when the prosthetic heart valve 110 is positioned within the native valve annulus and the anchor 210 is in contact with the outer surface of the heart. In other words, the positions of the prosthetic heart valve 110 and the anchor 220 are mostly fixed, and the entire length of the tether 226 from end to end is also fixed. As the tensioning member 400 lengthens, the distance between the prosthetic heart valve 110 and the anchor 210 remains substantially fixed, and the length of the tether 226 remains substantially fixed, resulting in increased tension on the tether 226 upon lengthening of the tensioning member 400.

In use, the tether 226 threaded through the assembled tensioning member 400 as described above, and then both ends of the tether 226 may be fixed to the prosthetic heart valve 110 and the anchor 210. The tensioning member 400 may initially be in the fully contracted state of FIG. 23H to allow for maximum tensioning of tether 226, although in other embodiments the tensioning member 400 may initially be in a condition that is longer than the fully contracted state. The prosthetic heart valve 110 and anchor 210 may be delivered in any suitable fashion. For example, a transapical procedure may be performed in which the prosthetic heart valve 110 is first implanted in the native valve annulus and the anchor 210 is then positioned in contact with the outer wall of the ventricle. On the other hand, a transseptal procedure may be performed in which the anchor 210 is first positioned in contact with the outer wall of the ventricle and the prosthetic heart valve 110 is next implanted in the native valve annulus. Regardless of the delivery route, the tensioning member 400 may then be adjusted by rotating the lead screw 455 to increase the length of the tensioning member 400 to tension the tether 226, although it should be understood that tension 226 can similarly be released by rotating the lead screw 455 in the opposite direction to decrease the length of the tensioning member 400. The lead screw 455 may be actuated by a catheter member with a distal end that passes through aperture 470 and mates with the head of the lead screw 455. Once the desired tension of tether 226 is reached, the catheter member may be removed from the tensioning device 400 and the tether 226 will remain at the desired tension due to the engagement of the lead screw 455 and nut 440 maintain the tensioning device 400 at the desired length. If it becomes necessary to adjust the tension of the tether 226 further, for example at a later date, the tensioning member 400 may simply be adjusted again to increase or decrease its length to change the tension on the tether 226.

In the embodiment of tensioning member 400 described above, the tether 226 may be effectively wrapped a single time around the tensioning member (i.e. the tether will have a single 180 degree turn at a top of the tensioning member 400 and a single 180 degree turn at the bottom of the tensioning member 400). However, in other embodiments, the tether 226 may be wrapped two or more times around the tensioning member 400. If the tether 226 is wrapped more times around the tensioning member 400, a given change in elongation of the tensioning member 400 will lead to a greater amount of tensioning of the tether 226 compared to if the tether 225 is wrapped fewer times around the tensioning member 400. In other words, if the tether 226 forms a single loop around the tensioning member 400, a given change in length of the tensioning member 400 will result in about the same change in effective length of the tether 226 between the prosthetic heart valve 110 and the anchor 210. However, if the tether 226 forms multiple loops around the tensioning member 400, that same given change in length of the tensioning member 400 will result in a larger change in effective length of the tether 226 between the prosthetic heart valve and the anchor 210. Also, while the mechanism of actuating (e.g. extending or contracting) the tensioning member 400 is described above as a lead screw 455 interacting with a complementary threaded nut 440, other actuation mechanisms may be suitable. For example, the first sliding component 410 and second base component 450 may be coupled in a substantially fluid-tight manner with an interior volume that may be accessed to inject or extract (e.g. via a valve that leads into the interior space) a fluid (e.g. a pressurized fluid) in order to force expansion or contraction of the tensioning member 400. If a fluid is used to expand the tensioning member 400, a biasing element, such as a spring, may couple the first sliding component 410 and the second base component 450 so that removal of fluid will tend to contract the tensioning member 400. Still other suitable actuation mechanisms may be suitable.

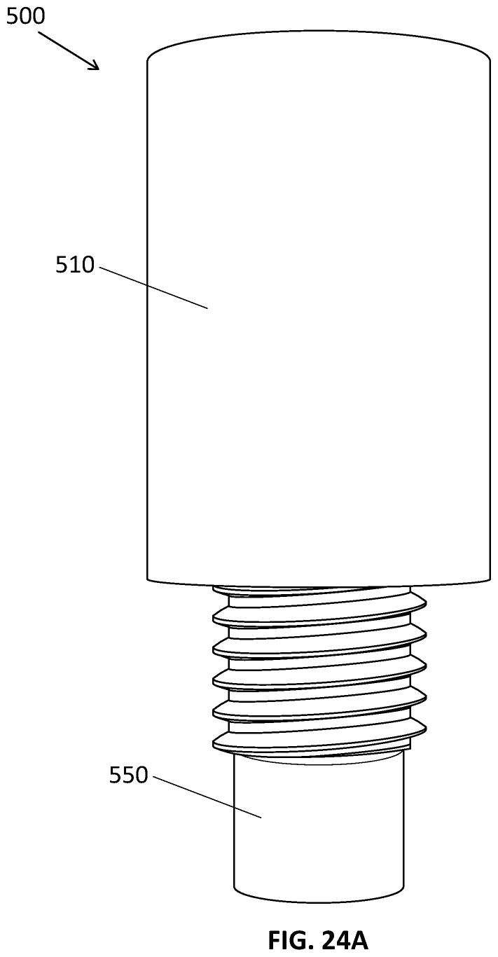

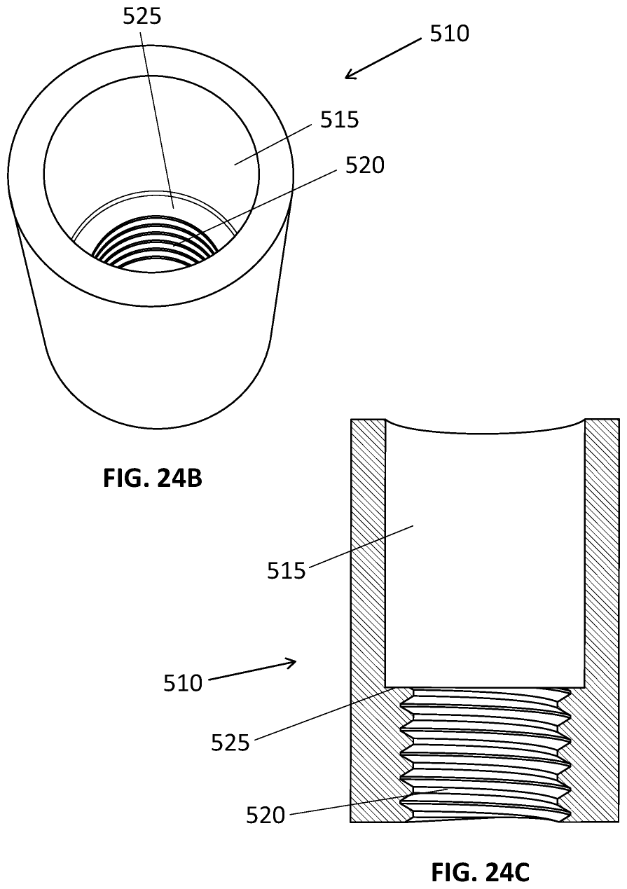

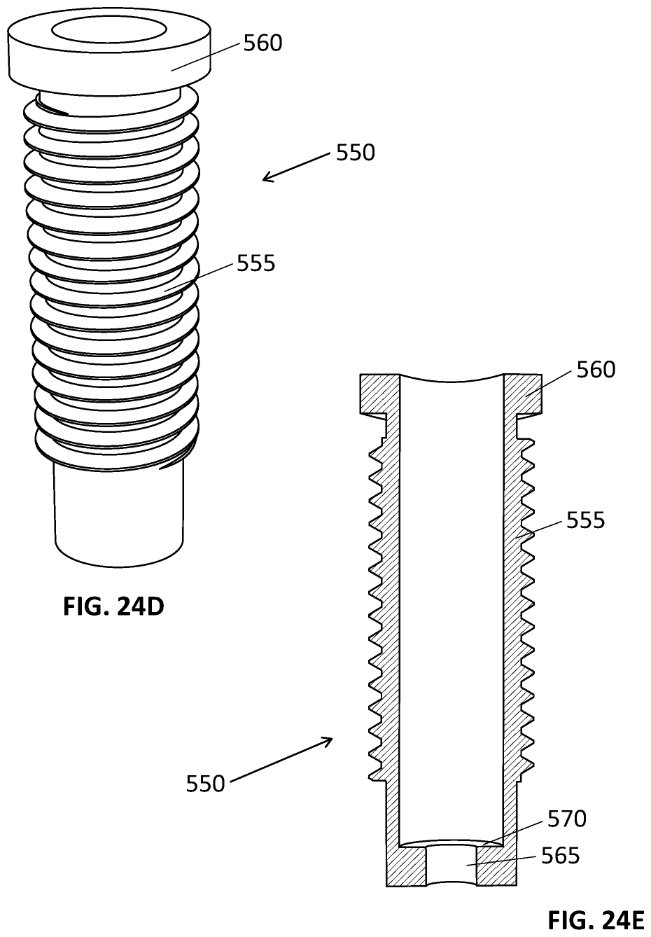

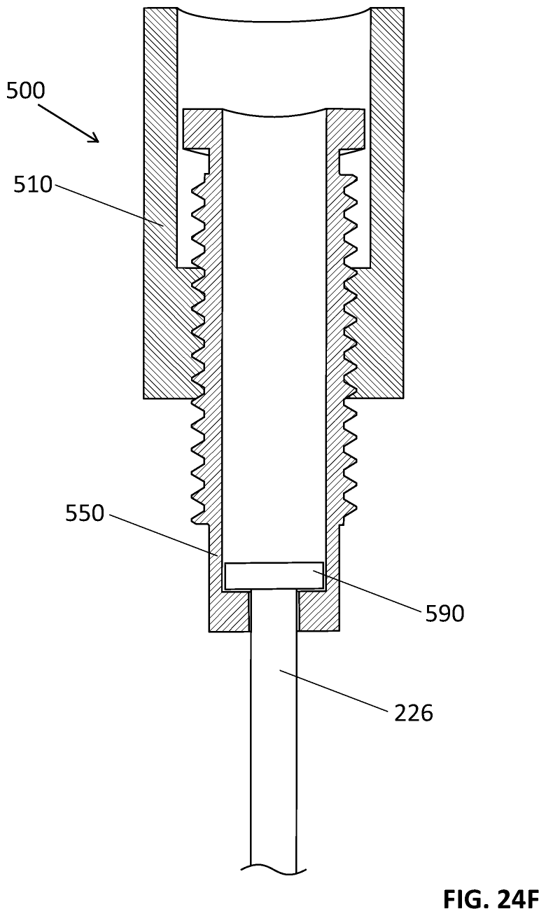

FIG. 24A is a front view of a tensioning member 500 according to another aspect of the disclosure. Generally, tensioning member 500 may include an outer housing 510 and a shaft member 550 received therein, the outer housing 510 and shaft member 550 adapted to translate relative to each other (e.g. via a general telescoping action).

FIG. 24B is a top perspective view of the outer housing 510, while FIG. 24C is a longitudinal cross-section of the outer housing 510. Generally, the outer housing 510 may have a cylindrical outer surface and a hollow interior volume. The hollow interior volume of the outer housing 510 may include a first portion 515 with a relatively large interior diameter, which may be smooth or unthreaded, and a second portion 520 with a relatively small interior diameter, which is preferably threaded, with an interior ledge 525 serving as the transition between the first portion 515 and the second portion 520. The axial ends of the outer housing 510 may be open so that a through-hole extends along the entire inner length of the outer housing 510.

FIG. 24D is a top perspective view of the shaft member 550, while FIG. 24E is a longitudinal cross-section of the shaft member 550. Generally, the shaft member 550 may be cylindrical with a generally cylindrical hollow interior volume. Shaft member 550 may include outer threading 555 along a portion of its length, although more or less threading may be provided than is shown in FIGS. 24D-E. The outer threading 555 may be adapted to engage with the inner threading of the second portion 520 of the outer housing 510, described in greater detail below. One end of the shaft member 550 may include a flange 560 having a generally cylindrical form with an outer diameter that is greater than the outer diameter of the remainder of the shaft member 550. As is described in greater detail below, the flange 560 may be sized to be smaller than the inner diameter of the first portion 515 of the outer housing 510, but greater than the inner diameter of the second portion 520 of the outer housing 510, to prevent the shaft member 550 from being “pulled through” the outer housing 510 during use. The end of shaft member 550 opposite the flange 560 may include an aperture 565, which may have an inner diameter smaller than the inner diameter of the remainder of the shaft member 550. An interior ledge or shoulder 570 may be formed where the inner diameter for the shaft member 550 transitions to aperture 565.

FIG. 24F is a cross-section of the tensioning member 500 in an assembled condition, with tether 226 coupled to the shaft member 550. In the illustrated embodiment, an end of the tether 226 is coupled to a button or ring 590. The ring 590 may be generally circular or cylindrical with an outer diameter about equal to or smaller than the inner diameter of the shaft member 550, but with an outer diameter larger than the inner diameter of aperture 565. With this configuration, ring 590 may abut shoulder 570, with the size difference preventing the ring 590 from pulling through the aperture 565. The tether 226 has an outer diameter about equal to or smaller than the inner diameter of the aperture 565, allowing the tether 226 to pass through the aperture 565. Preferably, the ring 590 is not fixedly coupled to the shaft member 550. With this configuration, while shaft member 550 rotates to drive the shaft member 550 into or out of the outer housing 510, the ring 590 and tether 226 may avoid rotation, which may undesirably torque the tether 226. The ring 590 and/or the inner surfaces of the shaft member 550 may have low-friction properties to facilitate the tether 226 avoiding rotation while the shaft member 550 rotates. Tether 226 may be coupled to ring 590 by any suitable mechanism, such as one or more fasteners such as sutures. It should be understood that ring 590 may take other shapes and forms besides a full ring. For example, ring 590 may instead have a rectangular shape with curved edges to allow it to rotate without torqueing the tether. Still other configurations that achieve the desired function of ring 590 may be appropriate.

In use, the outer housing 510 may be affixed to either the prosthetic heart valve 110 or the anchor 210. For example, in one embodiment the outer housing 510 may be positioned within or connected to the tether connecting or clamping portion 144 of the inner frame 140, so that the shaft member 550 will extend toward the anchor 210. In another embodiment, the outer housing 510 may be positioned within or connected to the anchor 210, for example at a center portion of the anchor 210, so that the shaft member 550 will extend toward the prosthetic heart valve 110. Although either configuration may be suitable for any delivery route, it may be preferable for the outer housing 510 to be coupled to the prosthetic heart valve 110 when a transseptal (e.g. transfemoral or transjugular) delivery is contemplated, and it may be preferable for the outer housing 510 to be coupled to the anchor 210 when a transapical delivery is contemplated. Prior to delivery, the tether 226 may be coupled to the shaft member 550 (similar to as shown in FIG. 24F), with the opposite end of the tether coupled to the anchor 210 (if the outer housing 510 is coupled to the prosthetic heart valve 110) or to the prosthetic heart valve 110 (if the outer housing 510 is coupled to the anchor 210). However, in other embodiments, the end of the tether 226 not coupled to the shaft member 550 may be free and connected to the anchor 210 or prosthetic heart valve 110 only after the delivery process begins. Both options are described below.

If the free ends of the tether 226 are coupled to the shaft member 550 and the prosthetic heart valve 110 (or anchor 210) prior to delivery, the shaft member 550 may be threaded to its maximum extent away from the housing 510, so that the tether 226 has its maximum slack. The prosthetic heart valve 110 and anchor 210 may be delivered in any suitable manner (e.g. transapically or transseptally) so that the prosthetic heart valve 110 is positioned within the native valve annulus, and the anchor 210 is in contact with or adjacent an exterior surface of the heart. In order to tension the tether 226, a device may be engaged with the flange 560 of the shaft member 550 in order to rotate the shaft member 550, advancing the shaft member 550 farther into the outer housing 510 (e.g. in the upward direction in the view of FIG. 24F). Engagement with the flange 560 may be accomplished by any suitable means. For example, although flange 560 is illustrated as having a continuous circular outer circumference, grooves or indents may be provided in that outer circumferential surface, for example two grooves/indents 180 degrees apart, 4 grooves/indents 90 degrees apart, etc. A tool may grip the flange 560 at the grooves/indents to facilitate rotation of the shaft member 550 relative to the outer housing 510. Similar grooves or indents may be additionally or alternatively provided on the opposite end of the shaft member 550 to allow for gripping and rotating the shaft member 550 at either end. If the outer housing 510 is fixed to the prosthetic heart valve 110, the tool to engage the flange 560 may be provided at or near an end of a catheter that is passed into the left atrium in a similar manner as described above in connection with the transseptal implantation of prosthetic heart valve 110. The tool may passed through the interior of the prosthetic heart valve 110 to access the flange 560, so that the tool may grip the flange 560 and rotate the flange 560 to draw the shaft member 550 away from the anchor 210 (in the upward direction in the view of FIG. 24F) to increase tension on the tether 226. The rotation of shaft member 550 may continue until the desired tension is reached. The interaction between the eternal threads of the shaft member 550 and the internal threads of the outer housing 510 may provide enough friction so that the tether 226 remains at the desired tension. In other words, the engagement between the shaft member 550 and the outer housing 510 may be such that the shaft member 550 and the outer housing 510 do not move (or do not significantly move) relative to one another in the absence of an intentionally applied rotational force to the shaft member 550. On the other hand, if the outer housing 510 is fixed to the anchor 210, the flange 560 of the shaft member 550 may be accessible from outside the heart, for example through a central hole in the portion of the anchor 210 facing away from the wall of the heart. A driver may be passed into the anchor 210 to rotate the shaft member 550 to tension the tether 226, in a manner generally similar to that described above.

In another embodiment, one end of the tether 226 remains free at the beginning of delivery, and is attached to the prosthetic heart valve 110 or anchor 210 only after delivery begins. For example, during a fully transseptal delivery, similar to that described in connection with FIGS. 11A-21B, the outer housing 510 may be fixed to the anchor 210 at the start of delivery. At the outset, the shaft member 550 may be positioned at a general mid-point so that the tension 226 can be either tensioned or slackened, although in other embodiments the shaft member 550 may be at other positions relative to the outer housing 510 to allow for introduction of a relatively higher amount of tension or slack during a later adjustment procedure. The anchor 210 may be delivered and positioned first, and then the prosthetic heart valve 110 may be delivered over the tether 226, using the tether 226 as a rail, until the prosthetic heart valve 110 is within the native valve annulus. The tether 226 may be tensioned (without the use of tensioning member 500) and then fixed to the prosthetic heart valve 110 at the desired tension. For example, the prosthetic heart valve 110 may include interiorly and upwardly extending barbs so that the prosthetic heart valve 110 may pass over the tether 226 in one direction toward the native heart valve, but not in the opposite direction. Other suitable mechanisms for tensioning the tether during this type of delivery are described in U.S. Patent Application No. 63/001,637, filed Mar. 30, 2020 and titled “Apparatus and Methods for Valve and Tether Fixation,” the disclosure of which is hereby incorporated by reference herein. Any remaining excess length of tether 226 extending proximally of the point of connection between the prosthetic heart valve 110 and the tether 226 may be cut, for example by a cautery tool. It should be understood that, in this particular example, the tensioning member 500 need not be used to tension the tether 226 during the initial delivery. However, if it becomes desirable to change the tension of the tether 226 after the initial delivery is complete (e.g. immediately after the excess length of the tether 226 is cut, or even days or weeks after the procedure is finished) the tensioning member 500 may be accessed in order to rotate the shaft member 550 to either increase or decrease the tension on the tether 226. This may be particularly useful if, for example, the tension on the tether 226 reduces as the anatomy and/or the components of the prosthetic heart valve system acclimate to the forces initially applied, which may introduce undesirable slack into the tether 226.

Tensioning member 500 may also be used in a transapical procedure in which one end of the tether 226 remains free until the final step(s) of the implantation. For example, prosthetic heart valve 110 may be implanted into a native valve annulus through a transapical puncture in the heart while the outer housing 510 is fixed to the tether connecting portion 144. The shaft member 550 may begin at a middle position relative to the outer housing 510 to allow for future tensioning or introduction of slack, although in other embodiments the shaft member 550 may be at other positions relative to the outer housing 510 to allow for introduction of a relatively higher amount of tension or slack during a later adjustment procedure. As the prosthetic heart valve 110 is positioned within the native heart valve annulus, the free end of the tether 226 remains accessible outside the heart by the user. An anchor 210 may be coupled to a tensioning device, and may be slid over the tether 226 into contact with an outer wall of the heart, with the anchor 210 being locked to the tether 226 while the tensioning device is coupled to the anchor 210. Examples of suitable tensioning devices are described in greater detail in U.S. Patent Application Publication Nos. 2016/0367368 and 2018/0028314, the disclosure of which is hereby incorporated by reference herein. Any excess length of tether 226 extending beyond the anchor 210 outside the heart may be cut, and the implantation may be concluded. Again, if it becomes desirable to change the tension of the tether 226 after the initial delivery is complete (e.g. immediately after the excess length of the tether 226 is cut, or even days or weeks after the procedure is finished) the tensioning member 500 may be accessed in order to rotate the shaft member 550 to either increase or decrease the tension on the tether 226. This may be completed, for example, by passing a catheter through the vasculature, into the heart, and through an interior of the prosthetic heart valve 110 to access the flange 560 of shaft member 550. As described above, a tool (e.g. at a distal end of the catheter), may engage the flange 560 and rotate the flange to move the shaft member 550 up or down relative to the outer housing 510. Further as noted above, the tether 226 may avoid corresponding rotation (and thus avoid undesirable torqueing) due to the interaction of the ring 590 with the shoulder 570 of the shaft member 550.

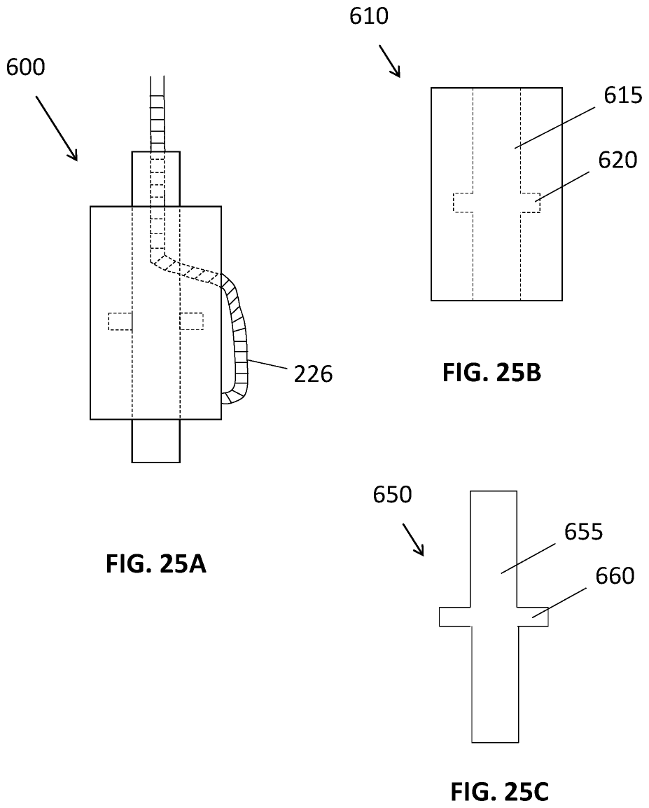

FIGS. 25A-C illustrate a tensioning member 600 according to another aspect of the disclosure. Generally, tensioning member 600 includes an outer housing 610 and an inner shaft 650. The outer housing 610 may be generally cylindrical, and may be mostly hollow to receive the inner shaft 650 therein. The inner shaft 650 may be substantially cylindrical main portion 655, with a cylindrical flange 660 extending outwardly therefrom. In an assembled condition of the tensioning member 600, the cylindrical main portion 655 may be received within a cylindrical opening 615 of the outer housing 610, and the cylindrical flange 660 may be received within a complementary cylindrical recess 620 of the outer housing 610. With this configuration, the inner shaft 650 may be generally freely rotatable with respect to the outer housing 610, but the inner shaft 650 may be axially fixed relative to the outer housing 610. It should be understood that other mechanisms and configurations may be suitable to allow for the inner shaft 650 and outer housing 610 to be rotatable relative to each other while being axially fixed relative to each other.

Referring to FIG. 25A, an end of the tether 226 may be fixed to a surface of the outer housing 610, for example via adhesives, welding, or any other suitable mechanism. A length of the tether 226 may extend along the outer surface of the outer housing 610, and may pass through an aperture (not separately labelled) in the outer housing 610, through an aperture (not separately labelled) in the inner shaft 650, and through a hollow interior of the inner shaft 650. The tether 226 may extend to a second free (or fixed) end.

Although not separately illustrated, the inner shaft 650 may include outer notches or grooves that interacted with inner notches or grooves of the outer housing 610, to allow for the rotational position of the outer housing 610 relative to the inner shaft 650 to be maintained in the absence of an intentionally applied rotational force. For example, the notches and/or grooves may be teeth-like and/or angled to have a ratchet-like configuration, in which rotation of the outer housing 610 is only possible in a single rotational direction relative to the inner shaft 650. With this configuration, as the outer housing 610 is rotated relative to the inner shaft 650, the tether 226 will wind around the outer surface of the outer housing 610 to tension the tether 226 (or otherwise reduce the effective length of the tether between its two ends). A helical groove or track (not illustrated) may be provided along the outer surface of the outer housing 610 so that, as the outer housing 610 rotates and causes the tether 226 to wind along the outer surface of the outer housing 610, the tether 226 winds within the groove or track.

The use of tensioning member 600 may be substantially similar to that described in connection with tensioning member 500, in which the inner shaft 650 is rotatably and axially fixed to the prosthetic heart valve 110 (for example to the tether connection portion 144) or the anchor 210, depending on the particular mode of delivery. The second end of the tether may be fixed to the other of the prosthetic heart valve 110 or anchor 210, with the entire tensioning being performed by rotating the outer housing 610 relative to the inner shaft 650. In other embodiments, the second end of the tether may be free and tensioned during implantation, without using tensioning member 600, and the second end of the tether is fixed to either the prosthetic heart valve 110 or the anchor 210 when the desired tension is achieved. During a later procedure (e.g. immediately after the implantation of days, weeks or more after the implantation), the outer housing 610 may be accessed and rotated to adjust the tension of the tether 226 by rotating the outer housing 610 about the inner shaft 650. As with other embodiments, the outer housing 610 may include exterior grooves or recesses, or similar structures, to facilitate a component in gripping the outer housing 610 to readily transmit torque to rotate the outer housing 610. In some embodiments, a release mechanism (e.g. a button) may be provided on tensioning member 600 to disengage the toothed/ratcheted/notched interface between the outer housing 610 and inner shaft 650 to allow for the tether 226 to unwind (after having been wound around the outer housing 610) to release an amount of tension on the tether 226.

According to one aspect of the disclosure, a prosthetic heart valve system comprises:

a prosthetic heart valve having an expandable stent and a prosthetic valve assembly disposed within the stent, the prosthetic valve assembly configured to allow blood to flow in a direction from an inflow end of the stent toward an outflow end of the stent and to substantially block blood from flowing from the outflow end of the stent toward the inflow end of the stent;

an anchor adapted to be disposed on or adjacent an epicardial surface of a heart of a patient;

a tether having a first end coupled to the anchor and a second end coupled to the prosthetic heart valve; and

a tensioning member disposed along an intermediate portion of the tether between the first end of the tether and the second end of the tether, the tether forming a loop around the tensioning member,

wherein the tensioning member is adjustable to increase or decrease a size of the loop to change an effective length of the tether between the prosthetic heart valve and the anchor; and/or

the tensioning member includes a shaft portion at least partially received within a base portion, the shaft portion being slideable into or out of the base portion; and/or

the base portion includes an externally threaded shaft, and the shaft portion includes an internally threaded nut that receives the externally threaded shaft, rotation of the externally threaded shaft relative to the internally threaded nut configured to cause the shaft portion to slide into or out of the base portion; and/or

the shaft portion includes a pulley along which the tether is routed, and the base portion includes a plurality of channels through which the tether is routed; and/or

the plurality of channels includes a first channel extending from a bottom surface of the base portion to a top surface of the base portion, and a second generally “U”-shaped channel having two outlets at the top surface.

According to another aspect of the disclosure, a prosthetic heart valve system comprises:

a prosthetic heart valve having an expandable stent and a prosthetic valve assembly disposed within the stent, the prosthetic valve assembly configured to allow blood to flow in a direction from an inflow end of the stent toward an outflow end of the stent and to substantially block blood from flowing from the outflow end of the stent toward the inflow end of the stent;

an anchor adapted to be disposed on or adjacent an epicardial surface of a heart of a patient;

a tensioning member having an externally threaded inner shaft received within an internally threaded outer housing; and

a tether having a first end coupled to the inner shaft, the inner shaft rotatable relative to the outer housing to: (i) translate the inner shaft into the outer housing to draw the tether closer to the outer housing; or (ii) translate the inner shaft out of the outer housing to move the tether away from the outer housing,

wherein in an implanted condition of the prosthetic heart valve system, either (i) the outer housing is fixed to the prosthetic heart valve and a second end of the tether is fixed to the anchor; or (ii) the outer housing is fixed to the anchor and the second end of the tether is fixed to the prosthetic heart valve; and/or

the inner shaft includes a main body and a flange at a first end of the main body, the flange having an outer diameter that is larger than an outer diameter of the main body; and/or

the outer housing includes a first portion axially spaced from a second portion, the first portion having an internal diameter larger than an internal diameter of the second portion, an interior ledge being formed at a transition between the first portion and the second portion; and/or

the outer diameter of the flange is larger than the internal diameter of the second portion of the outer housing, so that the flange can translate within the first portion of the outer housing but not the second portion of the outer housing; and/or

the inner shaft includes an aperture at a second end of the main body opposite the flange, the tether passing through the aperture into an interior portion of the inner shaft; and/or

the first end of the tether is coupled to a ring member having an outer diameter larger than an inner diameter of the aperture, so that the ring member cannot pull through the aperture; and/or

the ring member is rotatable relative to the inner shaft.

According to a further aspect of the disclosure, a prosthetic heart valve system comprises:

a prosthetic heart valve having an expandable stent and a prosthetic valve assembly disposed within the stent, the prosthetic valve assembly configured to allow blood to flow in a direction from an inflow end of the stent toward an outflow end of the stent and to substantially block blood from flowing from the outflow end of the stent toward the inflow end of the stent;

an anchor adapted to be disposed on or adjacent an epicardial surface of a heart of a patient;

a tether clamp having a first clamp portion, a second clamp portion, a base, and a cuff; and

a tether having a first end coupled to either the anchor or the prosthetic heart valve, the tether passing through an aperture in the base, between the first and second clamp portions, and through an aperture in the cuff,

wherein the first and second clamp portions have an unclamped condition in which clamping faces of the first and second clamp portions are spaced away from each other, and a clamped condition in which the clamping faces contact each other; and/or

the cuff includes an interior space configured to receive the first and second clamp portions when they are in the clamped condition, and when the first and second clamp portions are received within the cuff, the cuff prevents the first and second clamp portions from transitioning to the unclamped condition; and/or

the first clamp portion includes a first plurality of teeth formed by peaks and troughs, and the second clamp portion includes a second plurality of teeth formed by peaks and troughs, the peaks of the first plurality of teeth being received in the troughs of the second plurality of teeth when the first and second clamp portions are in the clamped condition; and/or

the cuff has a cylindrical body, the cuff including a central bar extending across a top surface of the cylindrical body, the aperture in the cuff formed within the central bar; and/or

in an assembled condition of the tether clamp, the first clamp portion is fixedly received within the base, and the second clamp portion is received within the base so that the second clamp portion is translatable toward and away from the first clamp portion; and/or

the first clamp portion includes at least two protrusions extending from a bottom surface thereof, the at least two protrusions received within at least two corresponding complementary shaped recesses within the base in the assembled condition of the tether clamp; and/or

the second clamp portion includes a “T”-shaped protrusion extending from a bottom surface thereof, the “T”-shaped protrusion received within a “T”-shaped recess within the base in the assembled condition of the tether clamp; and/or

in the assembled condition of the tether clamp, a narrow portion of the “T”-shaped protrusion is translatable within a narrow portion of the “T”-shaped recess.

Although the invention herein has been described with reference to particular embodiments, it is to be understood that these embodiments are merely illustrative of the principles and applications of the present invention. For example, although embodiments of prosthetic valves are described herein in the context of prosthetic mitral valves, the disclosure may substantially similarly apply to prosthetic tricuspid valves, with or without modifications. It is therefore to be understood that numerous modifications may be made to the illustrative embodiments and that other arrangements may be devised without departing from the spirit and scope of the present invention as defined by the appended claims.

1. A prosthetic heart valve system comprising:
a prosthetic heart valve having an expandable stent and a prosthetic valve assembly disposed within the stent, the prosthetic valve assembly configured to allow blood to flow in a direction from an inflow end of the stent toward an outflow end of the stent and to substantially block blood from flowing from the outflow end of the stent toward the inflow end of the stent;
an anchor adapted to be disposed on or adjacent an epicardial surface of a heart of a patient;
a tether having a first end coupled to the anchor and a second end coupled to the prosthetic heart valve; and
a tensioning member disposed along an intermediate portion of the tether between the first end of the tether and the second end of the tether, the tether forming a loop around the tensioning member,
wherein the tensioning member is adjustable to increase or decrease a size of the loop to change an effective length of the tether between the prosthetic heart valve and the anchor.

2. The prosthetic heart valve system of claim 1, wherein the tensioning member includes a shaft portion at least partially received within a base portion, the shaft portion being slideable into or out of the base portion.

3. The prosthetic heart valve system of claim 2, wherein the base portion includes an externally threaded shaft, and the shaft portion includes an internally threaded nut that receives the externally threaded shaft, rotation of the externally threaded shaft relative to the internally threaded nut configured to cause the shaft portion to slide into or out of the base portion.

4. The prosthetic heart valve system of claim 2, wherein the shaft portion includes a pulley along which the tether is routed, and the base portion includes a plurality of channels through which the tether is routed.

5. The prosthetic heart valve system of claim 4, wherein the plurality of channels includes a first channel extending from a bottom surface of the base portion to a top surface of the base portion, and a second generally “U”-shaped channel having two outlets at the top surface.

6. A prosthetic heart valve system comprising:
a prosthetic heart valve having an expandable stent and a prosthetic valve assembly disposed within the stent, the prosthetic valve assembly configured to allow blood to flow in a direction from an inflow end of the stent toward an outflow end of the stent and to substantially block blood from flowing from the outflow end of the stent toward the inflow end of the stent;
an anchor adapted to be disposed on or adjacent an epicardial surface of a heart of a patient;
a tensioning member having an externally threaded inner shaft received within an internally threaded outer housing; and
a tether having a first end coupled to the inner shaft, the inner shaft rotatable relative to the outer housing to: (i) translate the inner shaft into the outer housing to draw the tether closer to the outer housing; or (ii) translate the inner shaft out of the outer housing to move the tether away from the outer housing,
wherein in an implanted condition of the prosthetic heart valve system, either (i) the outer housing is fixed to the prosthetic heart valve and a second end of the tether is fixed to the anchor; or (ii) the outer housing is fixed to the anchor and the second end of the tether is fixed to the prosthetic heart valve.

7. The prosthetic heart valve system of claim 6, wherein the inner shaft includes a main body and a flange at a first end of the main body, the flange having an outer diameter that is larger than an outer diameter of the main body.

8. The prosthetic heart valve system of claim 7, wherein the outer housing includes a first portion axially spaced from a second portion, the first portion having an internal diameter larger than an internal diameter of the second portion, an interior ledge being formed at a transition between the first portion and the second portion.

9. The prosthetic heart valve system of claim 8, wherein the outer diameter of the flange is larger than the internal diameter of the second portion of the outer housing, so that the flange can translate within the first portion of the outer housing but not the second portion of the outer housing.

10. The prosthetic heart valve system of claim 7, wherein the inner shaft includes an aperture at a second end of the main body opposite the flange, the tether passing through the aperture into an interior portion of the inner shaft.

11. The prosthetic heart valve system of claim 10, wherein the first end of the tether is coupled to a ring member having an outer diameter larger than an inner diameter of the aperture, so that the ring member cannot pull through the aperture.

12. The prosthetic heart valve system of claim 11, wherein the ring member is rotatable relative to the inner shaft.

13. A prosthetic heart valve system comprising:
a prosthetic heart valve having an expandable stent and a prosthetic valve assembly disposed within the stent, the prosthetic valve assembly configured to allow blood to flow in a direction from an inflow end of the stent toward an outflow end of the stent and to substantially block blood from flowing from the outflow end of the stent toward the inflow end of the stent;
an anchor adapted to be disposed on or adjacent an epicardial surface of a heart of a patient;
a tether clamp having a first clamp portion, a second clamp portion, a base, and a cuff; and
a tether having a first end coupled to either the anchor or the prosthetic heart valve, the tether passing through an aperture in the base, between the first and second clamp portions, and through an aperture in the cuff,
wherein the first and second clamp portions have an unclamped condition in which clamping faces of the first and second clamp portions are spaced away from each other, and a clamped condition in which the clamping faces contact each other.

14. The prosthetic heart valve system of claim 13, wherein the cuff includes an interior space configured to receive the first and second clamp portions when they are in the clamped condition, and when the first and second clamp portions are received within the cuff, the cuff prevents the first and second clamp portions from transitioning to the unclamped condition.

15. The prosthetic heart valve system of claim 13, wherein the first clamp portion includes a first plurality of teeth formed by peaks and troughs, and the second clamp portion includes a second plurality of teeth formed by peaks and troughs, the peaks of the first plurality of teeth being received in the troughs of the second plurality of teeth when the first and second clamp portions are in the clamped condition.

16. The prosthetic heart valve system of claim 13, wherein the cuff has a cylindrical body, the cuff including a central bar extending across a top surface of the cylindrical body, the aperture in the cuff formed within the central bar.

17. The prosthetic heart valve system of claim 13, wherein in an assembled condition of the tether clamp, the first clamp portion is fixedly received within the base, and the second clamp portion is received within the base so that the second clamp portion is translatable toward and away from the first clamp portion.

18. The prosthetic heart valve system of claim 17, wherein the first clamp portion includes at least two protrusions extending from a bottom surface thereof, the at least two protrusions received within at least two corresponding complementary shaped recesses within the base in the assembled condition of the tether clamp.

19. The prosthetic heart valve of claim 18, wherein the second clamp portion includes a “T”-shaped protrusion extending from a bottom surface thereof, the “T”-shaped protrusion received within a “T”-shaped recess within the base in the assembled condition of the tether clamp.

20. The prosthetic heart valve of claim 19, wherein in the assembled condition of the tether clamp, a narrow portion of the “T”-shaped protrusion is translatable within a narrow portion of the “T”-shaped recess.
